(19) United States
(12) Patent Application Publication
(10) Pub. No.: US 2008/0075666 A1
(43) Pub. Date: Mar. 27, 2008

(54) Methods and compositions for treating diastolic dysfunction

(76) Inventors: Samuel C. Dudley JR., Atlanta, GA (US); Tai-Hwang Michael Fan, Roswell, GA (US); David G. Harrison, Atlanta, GA (US); Arshed Ali Quyyumi, Atlanta, GA (US)

Correspondence Address: THOMAS, KAYDEN, HORSTEMEYER & RISLEY, LLP, 600 GALLERIA PARKWAY, S.E., STE 1500, ATLANTA, GA, 30339-5994, US

(21) Appl. No.: 11/895,883

(22) Filed: Aug. 27, 2007

Related U.S. Application Data
(60) Provisional application No. 60/840,368, filed on Aug. 25, 2006.

(51) Int. Cl. A61K 31/519; A61K 31/40; A61K 38/44; A61P 9/00; G01N 33/15

(52) U.S. Cl. 424/9.2; 424/94.4; 514/250; 514/359

(57) ABSTRACT

Methods and compositions for treating and screening drugs for conditions are provided.

### CROSS-REFERENCE TO RELATED APPLICATION

This application claims the benefit of U.S. Provisional Patent entitled “METHODS AND COMPOSITIONS FOR TREATING DIASTOLIC DYSFUNCTION” Application No. 60/840,368 filed Aug. 25, 2006, the complete disclosures of which are incorporated herein by reference in there entirety.

### STATEMENT REGARDING FEDERALLY SPONSORED RESEARCH OR DEVELOPMENT

This invention was made with government support under NIH Grant No. NIH HL073753 awarded by the U.S. National Institutes of Health of the United States government. The government has certain rights in the invention.

### TECHNICAL FIELD

The present disclosure is generally related to methods and compositions for treating or preventing diastolic dysfunction, and in particular, methods and compositions for improving cardiac diastolic function.

### BACKGROUND

Heart failure is a major and growing public health problem in the United States. Approximately 5 million patients in this country have heart failure, and more than 550,000 patients are diagnosed with heart failure for the first time each year. The disorder is the primary reason for 12 to 15 million office visits and 6.5 million hospital days each year (Hunt et al., 2005). For many years, the syndrome of heart failure was considered to be synonymous with diminished contractility or reduced ejection fraction (EF). Over the past several years, however, there has been a growing appreciation that a large number of patients with heart failure have a relatively normal EF or preserved EF. This type of heart failure has been referred to as heart failure with normal EF, heart failure with preserved EF, or diastolic hear failure. Despite its high prevalence, there is currently no proven therapy for diastolic heart failure, in part due to a lack of understanding of the mechanisms that contribute to the development and progression of diastolic dysfunction.

Several recognized myocardial disorders are associated with diastolic heart failure, including restrictive cardiomyopathy, obstructive and non-obstructive hypertrophic cardiomyopathy, and infiltrative cardiomyopathies. The vast majority of patients with diastolic heart failure have a history of hypertension, and many of these patients have evidence of left ventricular hypertrophy on echocardiography. However, some patients who present with diastolic heart failure have no identifiable myocardial pathology.

Approximately half of all heart failure cases occur in patients with normal or preserved ejection fraction, making diastolic heart failure a substantial health problem. Defining diastolic dysfunction and choosing appropriate therapy has been hampered by a lack of mechanistic understanding of the condition.

Redfield et al. (2003) published the first-ever study to estimate the prevalence of left ventricular diastolic dysfunction in the community using comprehensive Doppler echocardiographic techniques. They found that diastolic dysfunction is very common and is often clinically silent. Furthermore, they found that diastolic dysfunction is associated with marked increases in all-cause mortality, with hazard ratios of 8.3 for mild diastolic dysfunction and 10.2 for moderate to severe diastolic dysfunction.

There has been no proven therapy to slow the progression of diastolic dysfunction, in part due to overall poor understanding of the mechanisms underlying diastolic dysfunction. Early diagnosis and treatment of preclinical diastolic dysfunction may prove to be a powerful strategy to reduce the incidence of heart failure.

### SUMMARY

Briefly described, embodiments of the present disclosure include methods of treating and/or preventing a condition (e.g., congestive heart failure, systolic heart failure, diastolic cardiac dysfunction, and diastolic heart failure, methods of treating and/or preventing nicotinamide adenine dinucleotide phosphate (NAD(P)H) oxidase activity, methods of treating and/or preventing the generation of reactive oxygen species (ROS), and the like.

One exemplary embodiment of a method of treating or preventing at least one condition, among others, includes: administering to a host in need of treatment a therapeutically effective amount of tetrahydrobiopterin (BH₄), wherein the condition is selected from: systolic heart failure, diastolic dysfunction, and diastolic heart failure.

One exemplary embodiment of a method of treating or preventing at least one condition, among others, includes: administering to a host in need of treatment a therapeutically effective amount of sepiapterin, wherein the condition is selected from: systolic heart failure, diastolic dysfunction, and diastolic heart failure.

One exemplary embodiment of a method of preserving diastolic function, among others, includes: administering to a host in need of treatment a therapeutically effective amount of tetrahydrobiopterin (BH₄).

One exemplary embodiment of a method of preserving diastolic function, among others, includes: administering to a host in need of treatment a therapeutically effective amount of tetrahydrobiopterin (BH₄).

One exemplary embodiment of a method of preserving diastolic function, among others, includes: administering to a host in need of treatment a therapeutically effective amount of ebselen and one or more antioxidants selected from superoxide dismutase, vitamins C and E, alpha lipoic acid, tempol and inhibitors of the NADPH oxidase.

One exemplary embodiment of a method of screening for compounds useful in treating or preventing at least one of: systolic heart failure, diastolic dysfunction, and diastolic heart failure, among others, includes: constructing an assay to measure generation of reactive oxygen species (ROS); contacting a host in need of treatment with a compound that prevents generation of ROS; detecting the effect of said compound on generation of ROS in said assay; and determining that the compound is a potential target, if said compound reduces or prevents ROS.

One exemplary embodiment of a method of screening for compounds useful in treating diastolic dysfunction, among others, includes: providing a DOCA-salt hypertensive mouse model, wherein the mouse has diastolic dysfunction, wherein the mouse has an intact systolic function, wherein the mouse is characterized by a rapid onset of diastolic dysfunction that is completely reversible, wherein the mouse is characterized by the absence of LV hypertrophy, and wherein the mouse is characterized by the absence of aortic or mitral regurgitation; detecting the effect of said compound on diastolic dysfunction; and determining that the compound is a potential target, if said compound reduces or prevents diastolic dysfunction.

Other systems, methods, features, and advantages of the present disclosure will be or will become apparent to one with skill in the art upon examination of the following drawings and detailed description. It is intended that all such additional systems, methods, features, and advantages be included within this description, be within the scope of the present disclosure, and be protected by the accompanying claims.

### BRIEF DESCRIPTION OF THE DRAWINGS

Many aspects of the disclosure can be better understood with reference to the following drawings. The components in the drawings are not necessarily to scale, emphasis instead being placed upon clearly illustrating the principles of the present disclosure.

The patent or patent application file contains at least one drawing executed in color. Copies of this patent or patent application publication with color drawing(s) will be provided by the Office upon request and payment of the necessary fee.

FIG. 1 illustrates a measurement of BP using the 8-Channel Non-Invasive Blood Pressure Monitor. The NIBP-8 system acquires the tail artery pulse and corresponding pressure signals through a pressurized sensor cuff during the transition between inflation and deflation of an occlusion cuff. A representative plethysmographic tracing from a conscious normal mouse is shown. In this example, the HR is 562 bpm and the systolic and diastolic BP is 114 mmHg and 78 mmHg, respectively. Animals are acclimated to the NIBP-8 environment before any actual measurements are undertaken.

FIG. 2 illustrates an assessment of diastolic function by LV inflow propagation velocity. The LV inflow propagation velocity (V_{P}) is determined by color M-mode Doppler. V_{P} is the slope connecting any isovelocity line (aliasing line) from the mitral valve tips to the LV apical region. In this example, the DOCA mouse has a decreased V_{P} than the sham-operated mouse (25 cm/sec vs. 45 cm/sec), indicating impaired LV relaxation.

FIG. 3 illustrates an assessment of diastolic function by mitral annulus longitudinal velocities. The mitral annulus longitudinal velocities are determined by pulsed-wave tissue Doppler echocardiography. Note that the sham-operated mouse has a normal E′ velocity of 5 cm/sec and a normal E′/A′ ratio of >1. In contrast, the DOCA mouse has a depressed E′ velocity (<2.5 cm/sec) and an abnormal E′/A′ ratio of <1, consistent with impaired LV relaxation.

FIG. 4 illustrates hemodynamic measurements in vivo. A 1.4-Fr Mikro-Tip catheter pressure transducer was inserted into the right common carotid artery and advanced into the LV for continuous LV pressure measurements. Each animal was allowed to stabilize for at least 10 min or until stable HR, LV systolic pressure, and maximal rate of pressure development (dP/dtₘₐₓ) were observed. As shown, the DOCA mice had a significantly elevated LVEDP compared to the SHAM mice. The dP/dtₘₐₓ/P (dP/dtₘₐₓ corrected by corresponding LV pressure), an index of LV systolic function, was statistically similar between the 2 groups. In contrast, the dP/dtₘᵢₙ/P (dP/dtₘᵢₙ corrected by corresponding LV pressure), an index of LV diastolic function, was significantly decreased in the DOCA mice compared to the SHAM mice. The findings are consistent with diastolic dysfunction with intact systolic function.

FIG. 5 illustrates a measurement of cardiac biopterin content. Panel A of FIG. 5: Cardiac biopterin content was measured using HPLC analysis and a differential oxidation method. Under acidic conditions, BH₄ and BH₂ are converted to fully oxidized biopterin. Under alkaline conditions, oxidation of BH₄ results in side chain cleavage and decomposition, while BH₂ is oxidized to biopterin. Thus, the net yield of biopterin from acidic oxidation (BH₄, BH₂ and biopterin) vs. alkaline oxidation (BH₂ and biopterin) can be used to determine the fraction of biopterin in the tetrahydro-form. Panels B & C of FIG. 5: Compared to the sham-operated mice, the DOCA mice had a significantly elevated level of oxidized biopterins and a decreased ratio of BH₄ to oxidized biopterins. The total cardiac biopterins contents (BH₄+BH₂+biopterin) were not significantly different between the DOCA group and the sham-operated group. Feeding BH₄ to DOCA mice significantly augmented their total cardiac biopterins contents and cardiac BH₄ levels. BH₄ feeding to DOCA mice also restored the BH₄/oxidized biopterins ratio to normal. *p<0.05 vs. SHAM group; §p<0.05 vs. DOCA group (n=3-6 in each group).

FIG. 6 illustrates basal NO levels in atria (LA & RA), atrial appendages (LM & RAA), and aortic tissues isolated from control and AF animals. NO levels were measured using a NO specific electrode. Data is presented as Mean ±SEM. *P<0.01. (Copyrighted by AHA).

FIG. 7 illustrates electron spin resonance (ESR) measurements of O₂ production. ESR spectra with the spin probe, CMH, were used to measure the production of intracellular O₂ in the right atrium (RA), right atrial appendage (RAA), left atrium (LA), and left atrial appendage (LAA) in AF (black bars) and control pigs (open bars). AF significantly increases intracellular O₂ production in the LA and LM (p<0.02 in each case). (Copyrighted by AHA).

FIG. 8 illustrates changes in NADPH oxidase and xanthine oxidase-mediated O₂ production as a result of AF. LA or LAA membrane preparations were made from control or AF pigs and exposed to either NADPH or hypoxanthine, substrates for the NADPH oxidase or xanthine oxidase, respectively. There was a statistically significant increase in O₂ production in LM of AF pigs when compared to control pigs (*, p=0.02). The trend was similar in the LA (p=0.06). Although appreciably smaller overall, hypoxanthine dependent O₂ production was increased in the LA (**, p=0.04) and LAA (#, p=0.01) with one week of AF. (Copyrighted by AHA).

FIG. 9 illustrates an assessment of LV diastolic function by echocardiography (leave is since it is in reference to a different example). Top panels of FIG. 9: LV inflow propagation velocity (V_{P}). Sham mice (left) have higher V_{P} than DOCA (right) mice. V_{P} is the slope connecting any isovelocity line (aliasing line) from the mitral valve tips to the LV apical region. Middle panels of FIG. 9: Tissue Doppler imaging. Sham mice (left) have a normal E′ velocity of 5 cm/sec and a normal E′/A′ ratio of >1. In contrast, the DOCA animals (right) have a depressed E′ velocity (<2.5 cm/sec) and an abnormal E′/A′ ratio of <1, consistent with impaired LV relaxation. Lower panels of FIG. 9: LV inflow velocities. Sham animals (left) have an E/A ratio of >1 and <2, which is normal. DOCA mice often show pseudonormalized E/A ratios suggesting the existence of moderate LV diastolic dysfunction.

FIG. 10 illustrates DOCA mice that have diastolic dysfunction by hemodynamic measures. FIG. 10A shows that LVEDP was significantly elevated in DOCA mice. FIG. 10B show dP/dtₘₐₓ/P (dP/dtₘₐₓ corrected by corresponding LV pressure), an index of LV systolic function was unchanged. FIG. 10C illustrates dP/dtₘᵢₙ/P (dP/dtₘᵢₙ corrected by corresponding LV pressure) reduced in DOCA mice. FIG. 10D illustrates the time constant of isovolumic LV pressure decline (τ) was prolonged in DOCA mice. *p<0.05 vs. sham group

FIG. 11 illustrates DOCA mice that have a steeper end-diastolic pressure-volume relation relationship. FIGS. 11A and 11B illustrate representative examples of LV pressure-volume loops obtained from DOCA and sham mice during transient IVC occlusion. The tracings show a progressive decline in chamber filling and stroke volume during IVC occlusion. Heart rate changed minimally during the few seconds required to obtain these data. The end-systolic pressure-volume relation (ESPVR) and end-diastolic pressure-volume relation (EDPVR) are shown. From the EDPVR, the chamber stiffness (k_{c}) and myocardial stiffness (kₘ) indices can be derived. C: On average, DOCA animals have a steeper EDPVR.

FIG. 12 illustrates DOCA mice that have less reduced BH₄. Panel A of FIG. 12: Cardiac biopterin content was measured using HPLC analysis and a differential oxidation method. Under acidic conditions, BH₄ and BH₂ are converted to fully oxidized biopterin. Under alkaline conditions, oxidation of BH₄ results in side chain cleavage and decomposition, while BH₂ is oxidized to biopterin. Panels B & C of FIG. 12: Compared to the sham-operated mice, the DOCA mice had a significantly elevated level of oxidized biopterins and a decreased ratio of BH₄ to oxidized biopterins. The total cardiac biopterins contents (BH₄+BH₂+biopterin) were not significantly different between the DOCA group and the sham-operated group. Feeding BH₄ to DOCA mice significantly augmented their total cardiac biopterins contents and cardiac BH₄ levels. BH₄ feeding to DOCA mice also restored the BH₄/oxidized biopterins ratio to normal. *p<0.05 vs. SHAM group; ^{§}p<0.05 vs. DOCA group (n=3-6 in each group).

FIG. 13 illustrates making a p22^{phox} KO mice. Panel A of FIG. 13 illustrates the targeting sequence used to create mice with LoxP sites flanking Exon 1 of p22^{phox}. Panel B of FIG. 13 illustrates PCR screening of tail clips from floxed p22^{phox} mice and siblings. PCR primers were designed to amplify LoxP3 or the region lacking LoxP3 in the WT mouse. The lower band represents the WT sequence and the highest band the sequence containing LoxP3. WT=Wild type. HT=Heterozygote. HO=Homozygote. Mice harboring the HO genotype for LoxP3 also showed PCR positive sequences for LoxP1 and LoxP2. These sequences are absent in WT mice because they rely on the presence of the Neo cassette.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Abbreviations and nomenclature, where employed, are deemed standard in the field and commonly used in professional journals such as those cited herein. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Embodiments of the present disclosure will employ, unless otherwise indicated, techniques of biochemistry, molecular biology, medicine, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

Prior to describing the various embodiments, the following definitions are provided and should be used unless otherwise indicated. Other terms may be defined elsewhere in the disclosure, as appropriate.

### DEFINITION

The term “treat”, “treating”, and “treatment” are an approach for obtaining beneficial or desired clinical results. Specifically, beneficial or desired clinical results include, but are not limited to, alleviation of symptoms, diminishment of extent of a condition, stabilization (e.g., not worsening) of a condition, preventing the condition from occurring in a host (e.g., human) that may be predisposed to the condition but does not yet experience or exhibit symptoms of the condition (prophylactic treatment), delaying or slowing of condition progression, amelioration or palliation of the condition, and remission (partial or total) whether detectable or undetectable. In addition, “treat”, “treating”, and “treatment” can also mean prolonging survival as compared to expected survival if not receiving treatment.

The term “condition” and “conditions” denote a state of health that can be related to: diastolic cardiac dysfunction, diastolic heart failure, and congestive heart failure The conditions that are discussed herein are to be included as conditions that can be treated by embodiments of the present disclosure.

The term “therapeutically effective amount” as used herein refers to that amount of the compound being administered which will relieve to some extent one or more of the symptoms of the condition being treated. In this regard, a therapeutically effective amount refers to that amount which has the effect of (1) reducing the severity of a condition, (2) inhibiting (that is, slowing to some extent, preferably stopping) the progression of the condition, (3) reversing the progression of the condition, (4) relieving to some extent (or, preferably, eliminating) one or more symptoms associated with a pathology related to or caused in part by the condition, and/or (5) preventing the chain of events downstream of an initial abnormal condition (that is, oxidative stress) which leads to the pathology. In reference to diastolic dysfunction (DD), a therapeutically effective amount refers to that amount which has the effect of (1) reducing the severity of DD, (2) inhibiting (that is, slowing to some extent, preferably stopping) the progression of DD, (3) reversing the progression of DD, (4) relieving to some extent (or, preferably, eliminating) one or more symptoms associated with a pathology related to or caused in part by DD, and/or (5) preventing the chain of events downstream of an initial abnormal condition (that is, oxidative stress) which leads to the pathology.

A “pharmaceutical composition” refers to a mixture of one or more of the compounds described herein, or pharmaceutically acceptable salts thereof, with other chemical components, such as physiologically acceptable carriers and excipients. One purpose of a pharmaceutical composition is to facilitate administration of a compound to an organism. The pharmaceutical composition can include one or more active agents. In an embodiment, the pharmaceutical composition consists essentially of an active agent disclosed herein (e.g., BH₄), and other components, including those designed to prevent oxidation in the gastrointestinal tract and those designed to increase the active BH₄ in tissues (e.g., sepiapterin), and inactive agents.

“Pharmaceutically acceptable salts” include, but are not limited to, the acid addition salts of compounds of the present disclosure (formed with free amino groups of the peptide) which are formed with inorganic acids (e.g., hydrochloric acid or phosphoric acids) and organic acids (e.g., acetic, oxalic, tartaric, or maleic acid). Salts formed with the free carboxyl groups may also be derived from inorganic bases (e.g., sodium, potassium, ammonium, calcium, or ferric hydroxides), and organic bases (e.g., isopropylamine, trimethylamine, 2-ethylamino-ethanol, histidine, and procaine).

The disclosed compounds may form salts that are also within the scope of this disclosure. Reference to a compound of any of the formulas herein is understood to include reference to salts thereof, unless otherwise indicated. The term “salt(s)”, as employed herein, denotes acidic and/or basic salts formed with inorganic and/or organic acids and bases. In addition, when a compound of the present disclosure contains both a basic moiety and an acidic moiety, zwitterions (“inner salts”) may be formed and are included within the term “salt(s)” as used herein. Pharmaceutically acceptable (e.g., non-toxic, physiologically acceptable) salts are preferred, although other salts are also useful, e.g., in isolation or purification steps which may be employed during preparation. Salts of the compounds of the present disclosure may be formed, for example, by reacting a compound of the present disclosure with an amount of acid or base, such as an equivalent amount, in a medium such as one in which the salt precipitates or in an aqueous medium followed by lyophilization.

The disclosed compounds that contain a basic moiety may form salts with a variety of organic and inorganic acids. Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides (formed with hydrochloric acid), hydrobromides (formed with hydrogen bromide), hydroiodides, 2-hydroxyethanesulfonates, lactates, maleates (formed with maleic acid), methanesulfonates (formed with methanesulfonic acid), 2-naphthalenesulfonates, nicotinates, nitrates, oxalates, pectinates, persulfates, 3-phenylpropionates, phosphates, picrates, pivalates, propionates, salicylates, succinates, sulfates (such as those formed with sulfuric acid), sulfonates (such as those mentioned herein), tartrates, thiocyanates, toluenesulfonates such as tosylates, undecanoates, and the like.

The disclosed compounds that contain an acidic moiety may form salts with a variety of organic and inorganic bases. Exemplary basic salts include ammonium salts, alkali metal salts such as sodium, lithium, and potassium salts, alkaline earth metal salts such as calcium and magnesium salts, salts with organic bases (for example, organic amines) such as benzathines, dicyclohexylamines, hydrabamines (formed with N,N-bis(dehydroabietyl)ethylenediamine), N-methyl-D-glucamines, N-methyl-D-glucamides, t-butyl amines, and salts with amino acids such as arginine, lysine, and the like.

Basic nitrogen-containing groups may be quaternized with agents such as lower alkyl halides (e.g., methyl, ethyl, propyl, and butyl chlorides, bromides and iodides), dialkyl sulfates (e.g., dimethyl, diethyl, dibutyl, and diamyl sulfates), long chain halides (e.g., decyl, lauryl, myristyl and stearyl chlorides, bromides and iodides), aralkyl halides (e.g., benzyl and phenethyl bromides), and others.

Solvates of the compounds of the disclosure are also contemplated herein. Solvates of the compounds are preferably hydrates.

To the extent that the disclosed compounds, and salts thereof, may exist in their tautomeric form, all such tautomeric forms are contemplated herein as part of the present disclosure.

As used herein, a “pharmaceutically acceptable carrier” refers to a carrier or diluent that does not cause significant irritation to an organism and does not abrogate the biological activity and properties of the administered compound.

An “excipient” refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

The term “prodrug” refers to an agent that is converted into a biologically active form in vivo. Prodrugs are often useful because, in some situations, they may be easier to administer than the parent compound. They may, for instance, be bioavailable by oral administration whereas the parent compound is not. The prodrug may also have improved solubility in pharmaceutical compositions over the parent drug. A prodrug may be converted into the parent drug by various mechanisms, including enzymatic processes and metabolic hydrolysis. Harper, N. J. (1962). Drug Latentiation in Jucker, ed. Progress in Drug Research, 4:221-294; Morozowich et al. (1977). Application of Physical Organic Principles to Prodrug Design in E. B. Roche ed. Design of Biopharmaceutical Properties through Prodrugs and Analogs, APhA; Acad. Pharm. Sci.; E. B. Roche, ed. (1977). Bioreversible Carriers in Drug in Drug Design, Theory and Application, APhA; H. Bundgaard, ed. (1985) Design of Prodrugs, Elsevier; Wang et al. (1999) Prodrug approaches to the improved delivery of peptide drug, Curr. Pharm. Design. 5(4):265-287; Pauletti et al. (1997). Improvement in peptide bioavailability: Peptidomimetics and Prodrug Strategies, Adv. Drug. Delivery Rev. 27:235-256; Mizen et al. (1998). The Use of Esters as Prodrugs for Oral Delivery of β-Lactam antibiotics, Pharm. Biotech. 11:345-365; Gaignault et al. (1996). Designing Prodrugs and Bioprecursors I. Carrier Prodrugs, Pract. Med. Chem. 671-696; M. Asgharnejad (2000). Improving Oral Drug Transport Via Prodrugs, in G. L. Amidon, P. I. Lee and E. M. Topp, Eds., Transport Processes in Pharmaceutical Systems, Marcell Dekker, p. 185-218; Balant et al. (1990) Prodrugs for the improvement of drug absorption via different routes of administration, Eur. J. Drug Metab. Pharmacokinet., 15(2): 143-53; Balimane and Sinko (1999). Involvement of multiple transporters in the oral absorption of nucleoside analogues, Adv. Drug Delivery Rev., 39(1-3):183-209; Browne (1997). Fosphenyloin (Cerebyx), Clin. Neuropharmacol. 20(1): 1-12; Bundgaard (1979). Bioreversible derivatization of drugs—principle and applicability to improve the therapeutic effects of drugs, Arch. Pharm. Chemi. 86(1): 1-39; H. Bundgaard, ed. (1985) Design of Prodrugs, New York: Elsevier; Fleisher et al. (1996). Improved oral drug delivery: solubility limitations overcome by the use of prodrugs, Adv. Drug Delivery Rev. 19(2): 115-130; Fleisher et al. (1985). Design of prodrugs for improved gastrointestinal absorption by intestinal enzyme targeting, Methods Enzymol. 112: 360-81; Farquhar D, et al. (1983). Biologically Reversible Phosphate-Protective Groups, J. Pharm. Sci., 72(3): 324-325; Han, H. K. et al. (2000). Targeted prodrug design to optimize drug delivery, AAPS Pharm Sci., 2(1): E6; Sadzuka Y. (2000). Effective prodrug liposome and conversion to active metabolite, Curr. Drug Metab., 1(1):31-48; D. M. Lambert (2000) Rationale and applications of lipids as prodrug carriers, Eur. J. Pharm. Sci., 11 Suppl 2:S15-27; Wang, W. et al. (1999) Prodrug approaches to the improved delivery of peptide drugs. Curr. Pharm. Des., 5(4):265-87.

Tetrahydrobiopterin (BH₄) refers to the natural and the unnatural forms of tetrahydrobiopterin, pharmaceutically compatible salts thereof and any mixtures of the isomers and the salts. In addition, embodiment of the present disclosure include precursors of tetrahydrobiopterin such as, but not limited to, 7,8-dihydrobiopterin, sepiapterin, biopterin, and other pterins that can generate BH₄ in vivo.

“Heart failure”, “congestive heart failure (CHF)”, and “congestive cardiac failure (CCF), define a condition that can result from any structural or functional cardiac disorder that impairs the ability of the heart to fill with or pump a sufficient amount of blood through the body.

“Hypertension” (also referred to as “arterial hypertension”) is a medical condition in which the blood pressure is chronically elevated. Hypertension is considered to be present when a person's systolic blood pressure is consistently 140 mmHg or greater, and/or their diastolic blood pressure is consistently 90 mmHg or greater.

“Diastolic dysfunction” refers to an abnormality in the heart's (i.e., left ventricle's) filling during diastole. Diastole is that phase of the cardiac cycle when the heart (i.e., ventricle) is not contracting but is actually relaxed and filling with blood that is being returned to it, either from the body (into right ventricle) or from the lungs (into left ventricle).

The term “dietary supplement” refers to materials defined as dietary supplements in Section 3 of the Dietary Supplement Health and Education Act of 1994, Public Law 103-417, Oct. 25, 1994. A dietary supplement is a product taken by mouth that contains a “dietary ingredient” intended to supplement the diet. The “dietary ingredients” include the one or more of the compounds described in embodiments of the present disclosure and optionally one or more other dietary ingredients such as vitamins, minerals, herbs or other botanicals, amino acids, and substances such as enzymes, organ tissues, glandulars, and metabolites. Dietary supplements can also be extracts or concentrates, and may be found in many forms such as tablets, capsules, softgels, gelcaps, liquids, or powders.

As used herein, a “food product formulated for human consumption” is a composition intended for ingestion by a human being.

As used herein, the term “food”, whether for human or nonhuman animals, includes compositions of any texture, consistency, moisture content, and the like, including both solid and nonsolid (for example, emulsions, suspensions, gels, and liquids) foods.

The terms “including”, “such as”, “for example” and the like are intended to refer to exemplary embodiments and not to limit the scope of the present disclosure.

As used herein, the term “host” or “organism” includes humans, mammals (e.g., cats, dogs, horses, etc.), living cells, and other living organisms. A living organism can be as simple as, for example, a single eukaryotic cell or as complex as a mammal. Typical hosts to which embodiments of the present disclosure may be administered will be mammals, particularly primates, especially humans. For veterinary applications, a wide variety of subjects will be suitable, e.g., livestock such as cattle, sheep, goats, cows, swine, and the like; poultry such as chickens, ducks, geese, turkeys, and the like; and domesticated animals particularly pets such as dogs and cats. For diagnostic or research applications, a wide variety of mammals will be suitable subjects, including rodents (e.g., mice, rats, hamsters), rabbits, primates, and swine such as inbred pigs and the like. Additionally, for in vitro applications, such as in vitro diagnostic and research applications, body fluids and cell samples of the above subjects will be suitable for use, such as mammalian (particularly primate such as human) blood, urine, or tissue samples, or blood, urine, or tissue samples of the animals mentioned for veterinary applications.

The term “consisting essentially of” is intended to refer to formulations that include the active component (e.g., BH₄) with other non-active components.

General Discussion

Quantitative Assessment of Diastolic Function

Diastole is often divided into 4 phases: isovolumic relaxation, early rapid filling phase, diastasis, and atrial contraction. After closure of the aortic valve, left ventricular (LV) pressure declines without a change in LV volume until left atrial (LA) pressure exceeds LV pressure and opens the mitral valve. This phase is measured by the isovolumic relaxation time (IVRT). The early rapid filling phase is driven by the atrioventricular pressure gradient across the mitral valve. Diastasis describes the period during which LV pressure is in equilibration with LA pressure. The contribution to LV filling by atrial contraction depends on ventricular diastolic pressure and stiffness and on atrial contractility.

Diastolic dysfunction can be characterized as mild, in which left ventricular relaxation is slowed; moderate, in which left ventricular relaxation is slowed and left atrial pressure is increased; and severe, in which left ventricular relaxation is slowed, left atrial pressure is increased, and left ventricular compliance (elasticity) is compromised.

While invasive hemodynamics remains a gold standard and is used in results discussed herein, ultrasound methods are used more frequently, allow for easy serial determinations, and can therefore also be used. The common noninvasive method for diagnosing diastolic dysfunction is through conventional pulsed-wave Doppler echocardiography by measuring the blood flow velocity through the mitral valve. In normal diastolic function, most blood flows through the mitral valve when the LV relaxes (known in echocardiography as the E wave, for early diastolic filling); then additional blood is pumped through the valve when LA contracts during late diastole (known as the A wave, for atrial contraction). For adult human hearts with normal diastolic function, the ratio of maximal E wave velocity to maximal A wave velocity is greater than 1 and less than 2. In mild diastolic dysfunction, there is less driving force for blood flow through the mitral valve during early diastole, and the E wave velocity decreases, resulting in a diminished E/A ratio of less than 1. In moderate diastolic dysfunction, a moderate rise in LA pressure causes less blood to be pumped from the atrium, therefore causing the A wave velocity to decrease, resulting in a pseudonormal E/A ratio of greater than 1 and less than 2. In severe diastolic dysfunction, high LA pressure accentuates the early LA-LV pressure gradient and causes the mitral valve to open earlier and at a higher crossover pressure. This results in rapid acceleration and deceleration of E wave velocity and a markedly diminished A wave velocity, leading to an abnormally high E/A ratio of greater than 2. Because the pseudonormal E/A pattern in moderate diastolic dysfunction cannot be readily differentiated from the true normal, the utility of conventional pulsed-wave Doppler for diagnosing diastolic dysfunction is limited when used in isolation.

Tissue Doppler Imaging

Tissue Doppler imaging (TDI) is a relatively new echocardiographic technique that is increasingly gaining popularity as a diagnostic tool for diastolic dysfunction. It has been shown to be relatively insensitive to preload, and is therefore particularly helpful in differentiating normal from pseudonormal filling pattern. TDI employs the Doppler principle to measure the velocity of myocardial segments and other cardiac structures. Impairment of longitudinal myocardial fiber motion is a sensitive marker of early myocardial dysfunction and ischemia. TDI allows quantitative measurement of long-axis ventricular function. Mitral annulus velocity in diastole is reflective of changes in velocity for the LV long axis. In normal hearts, the long axis and circumferential motion is approximately the same. By recording mitral annulus motion from the apex, the effect of myocardial translation is minimized. A typical spectral pattern will demonstrate a single systolic velocity toward the LV centroid (Sm), and two signals away from the centroid during early and late diastole. With abnormal active relaxation, mitral annulus velocity during early diastole (E′) is decreased while mitral annulus velocity during late diastole (A′) is increased, resulting in a lowered E′/A′ ratio. In restrictive cardiomyopathy, both the E′ and A′ are severely blunted. In contrast, the mitral annulus velocity is preserved in constrictive pericarditis. TDI has recently been validated as a reliable tool in the evaluation of diastolic dysfunction in mice.

Left Ventricular Inflow Propagation Velocity by Color M-Mode Doppler

Left ventricular inflow propagation velocity (V_{P}) by color M-mode Doppler is a preload insensitive index of LV relaxation. It has been shown to correlate well with the time constant of isovolumic relaxation (τ), both in animals and humans. In anesthetized dogs, V_{P} has proved to be independent of LA pressure and heart rate. More recently, V_{P} has also been shown to reflect changes in myocardial relaxation in mice with genetically altered levels of phospholamban. Color M-mode Doppler differs from conventional pulsed-wave Doppler in that it allows the acquisition of spatial information, in addition to velocity and time information. From the apical 4-chamber view, color flow Doppler is activated. Adjustments are made to obtain the longest column of color flow from the mitral valve tips to the LV apex. The M-mode cursor is positioned through the center of the color flow to run parallel with the direction of the mitral inflow. After maximizing sweep speed and appropriate shifting of the color flow map baselines, color M-mode images are acquired. The V_{P} is measured off-line with commercially available software (Xcelera, Philips Medical Systems) as the slope of the first aliasing line from the mitral valve tips to the LV apical region. In humans, the V_{P} is greater than 45 cm/s for normal adults and less than 45 cm/s in patients with diastolic dysfunction. In our laboratory, we have found the V_{P} values of normal mice to be quite comparable to that of normal humans.

Potential Mechanisms of Diastolic Dysfunction and its Progression

Several hypotheses have been made to explain the molecular mechanism behind diastolic dysfunction. In one hypothesis, cardiomyocytes have impaired abilities to relax because of problems with storage and transport of calcium, the ion responsible for muscle contraction. In other hypotheses, changes in the extracellular matrix around cardiomyocytes cause fibrosis that changes tissue elasticity, or changes in NO ^{production alter relaxation properties of the heart.}

Discussion

Embodiments of the present disclosure include methods of treating and/or preventing a condition (e.g., congestive heart failure, systolic heart failure, diastolic cardiac dysfunction, and diastolic heart failure). In particular, embodiments of the present disclosure include methods of treating and/or preventing diastolic dysfunction or diastolic heart failure. In addition, embodiments of the present disclosure include methods of treating and/or preventing nicotinamide adenine dinucleotide phosphate (NAD(P)H) oxidase activity. Furthermore, embodiments of the present disclosure include methods of treating and/or preventing the generation of reactive oxygen species (ROS).

Embodiments of the present disclosure can be used for the early diagnosis, treatment, and/or prevention of preclinical diastolic dysfunction, which may prove to be a powerful strategy to reduce the incidence of heart failure.

In particular, a murine model of diastolic dysfunction was developed using mice with deoxycorticosterone acetate (DOCA)-salt induced hypertension (see Examples). It was observed that dietary supplementation with tetrahydrobiopterin (BH₄) improves diastolic relaxation in these animals.

As mentioned above, embodiments of the present disclosure include methods of treating and/or preventing a condition such as, but not limited to, congestive heart failure, diastolic heart failure, diastolic cardiac dysfunction, and diastolic heart failure, by administering a composition or pharmaceutical composition to a host. The composition or pharmaceutical composition includes a tetrahydrobiopterin, derivatives thereof, pharmaceutically acceptable salts thereof, prodrugs, or combinations thereof. In an embodiment, the composition or pharmaceutical composition can consist essentially of tetrahydrobiopterin, derivatives thereof, pharmaceutically acceptable salts thereof, prodrugs, or combinations thereof, and nonactive agents or components designed to minimize oxidation during delivery.

In another embodiment, the composition or pharmaceutical composition can include tetrahydrobiopterin, sepiapterin, ebselen and other antioxidants such as, but not limited to, superoxide dismutase, vitamins C and E, alpha lipoic acid, tempol, and inhibitors of the NADPH oxidase, derivatives of each, pharmaceutically acceptable salts of each, prodrugs, and combinations thereof.

In another embodiment, the composition or pharmaceutical composition can consist essentially of one or more of the following: tetrahydrobiopterin, sepiapterin, ebselen and other antioxidants such as, but not limited to, superoxide dismutase, vitamins C and E, alpha lipoic acid, tempol, and inhibitors of the NADPH oxidase, derivatives of each, pharmaceutically acceptable salts of each, prodrugs, and combinations thereof, and nonactive agents or components.

In addition, embodiments of the present disclosure include methods of treating and/or preventing: nicotinamide adenine dinucleotide phosphate (NAD(P)H) oxidase activity, reactive oxygen species (ROS), and combinations thereof, by administering one of the embodiments of the compositions or pharmaceutical compositions described above.

In another embodiment, the present disclosure can include the use of ACE inhibitors, angiotensin receptor blockers, peroxisome proliferator-activated receptor agonists, and statins, along with embodiments of the compositions, pharmaceutical compositions, dietary supplements, and the like.

Pharmaceutical Compositions

Pharmaceutical compositions and dosage forms of the disclosure include a pharmaceutically acceptable salt of disclosed or a pharmaceutically acceptable polymorph, solvate, hydrate, dehydrate, co-crystal, anhydrous, or amorphous form thereof. Specific salts of disclosed compounds include, but are not limited to, sodium, lithium, potassium salts, and hydrates thereof.

Pharmaceutical compositions and unit dosage forms of the disclosure typically also include one or more pharmaceutically acceptable excipients or diluents. Advantages provided by specific compounds of the disclosure, such as, but not limited to, increased solubility and/or enhanced flow, purity, or stability (e.g., hygroscopicity) characteristics can make them better suited for pharmaceutical formulation and/or administration to patients than the prior art.

Pharmaceutical unit dosage forms of the compounds of this disclosure are suitable for oral, mucosal (e.g., nasal, sublingual, vaginal, buccal, or rectal), parenteral (e.g., intramuscular, subcutaneous, intravenous, intraarterial, or bolus injection), topical, or transdermal administration to a patient. Examples of dosage forms include, but are not limited to: tablets; caplets; capsules, such as hard gelatin capsules and soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The composition, shape, and type of dosage forms of the compositions of the disclosure will typically vary depending on their use. For example, a dosage form used in the acute treatment of a disease or disorder may contain larger amounts of the active ingredient, for example the disclosed compounds or combinations thereof, than a dosage form used in the chronic treatment of the same disease or disorder. Similarly, a parenteral dosage form may contain smaller amounts of the active ingredient than an oral dosage form used to treat the same disease or disorder. These and other ways in which specific dosage forms encompassed by this disclosure will vary from one another will be readily apparent to those skilled in the art. See, e.g., Remington's Pharmaceutical Sciences, 18th ed., Mack Publishing, Easton, Pa. (1990).

Typical pharmaceutical compositions and dosage forms comprise one or more excipients. Suitable excipients are well known to those skilled in the art of pharmacy or pharmaceutics, and non-limiting examples of suitable excipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets or capsules may contain excipients not suited for use in parenteral dosage forms. The suitability of a particular excipient may also depend on the specific active ingredients in the dosage form. For example, the decomposition of some active ingredients can be accelerated by some excipients such as lactose, or when exposed to water. Active ingredients that comprise primary or secondary amines are particularly susceptible to such accelerated decomposition.

The disclosure further encompasses pharmaceutical compositions and dosage forms that include one or more compounds that reduce the rate by which an active ingredient will decompose. Such compounds, which are referred to herein as “stabilizers,” include, but are not limited to, antioxidants such as ascorbic acid, pH buffers, or salt buffers. In addition, pharmaceutical compositions or dosage forms of the disclosure may contain one or more solubility modulators, such as sodium chloride, sodium sulfate, sodium or potassium phosphate or organic acids. A specific solubility modulator is tartaric acid.

Like the amounts and types of excipients, the amounts and specific type of active ingredient in a dosage form may differ depending on factors such as, but not limited to, the route by which it is to be administered to patients. However, typical dosage forms of the compounds of the disclosure comprise a pharmaceutically acceptable salt, or a pharmaceutically acceptable polymorph, solvate, hydrate, dehydrate, co-crystal, anhydrous, or amorphous form thereof, in an amount of from about 10 mg to about 1000 mg, preferably in an amount of from about 25 mg to about 750 mg, and more preferably in an amount of from 50 mg to 500 mg.

Additionally, the compounds and/or compositions can be delivered using lipid- or polymer-based nanoparticles. For example, the nanoparticles can be designed to improve the pharmacological and therapeutic properties of drugs administered parenterally (Science. 303 (5665):1818-22 (2004)).

Oral Dosage Forms

Pharmaceutical compositions of the disclosure that are suitable for oral administration can be presented as discrete dosage forms, such as, but not limited to, tablets (including without limitation scored or coated tablets), pills, caplets, capsules, chewable tablets, powder packets, cachets, troches, wafers, aerosol sprays, or liquids, such as but not limited to, syrups, elixirs, solutions or suspensions in an aqueous liquid, a non-aqueous liquid, an oil-in-water emulsion, or a water-in-oil emulsion. Such compositions contain a predetermined amount of the pharmaceutically acceptable salt of the disclosed compounds, and may be prepared by methods of pharmacy well known to those skilled in the art. See generally, Remington's Pharmaceutical Sciences, 18th ed., Mack Publishing, Easton, Pa. (1990).

Typical oral dosage forms of the compositions of the disclosure are prepared by combining the pharmaceutically acceptable salt of disclosed compounds in an intimate admixture with at least one excipient according to conventional pharmaceutical compounding techniques. Excipients can take a wide variety of forms depending on the form of the composition desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, microcrystalline cellulose, kaolin, diluents, granulating agents, lubricants, binders, and disintegrating agents.

Due to their ease of administration, tablets and capsules represent the most advantageous solid oral dosage unit forms, in which case solid pharmaceutical excipients are used. If desired, tablets can be coated by standard aqueous or nonaqueous techniques. These dosage forms can be prepared by any of the methods of pharmacy. In general, pharmaceutical compositions and dosage forms are prepared by uniformly and intimately admixing the active ingredient(s) with liquid carriers, finely divided solid carriers, or both, and then shaping the product into the desired presentation if necessary.

For example, a tablet can be prepared by compression or molding. Compressed tablets can be prepared by compressing in a suitable machine the active ingredient(s) in a free-flowing form, such as a powder or granules, optionally mixed with one or more excipients. Molded tablets can be made by molding in a suitable machine a mixture of the powdered compound moistened with an inert liquid diluent.

Examples of excipients that can be used in oral dosage forms of the disclosure include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Suitable forms of microcrystalline cellulose include, but are not limited to, the materials sold as AVICEL-PH-101, AVICEL-PH-103 AVICEL RC-581, and AVICEL-PH-105 (available from FMC Corporation, American Viscose Division, Avicel Sales, Marcus Hook, Pa., U.S.A.), and mixtures thereof. An exemplary suitable binder is a mixture of microcrystalline cellulose and sodium carboxymethyl cellulose sold as AVICEL RC-581. Suitable anhydrous or low moisture excipients or additives include AVICEL-PH-103™ and Starch 1500 LM.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the disclosure is typically present in from about 50 to about 99 weight percent of the pharmaceutical composition or dosage form.

Disintegrants are used in the compositions of the disclosure to provide tablets that disintegrate when exposed to an aqueous environment. Tablets that contain too much disintegrant may swell, crack, or disintegrate in storage, while those that contain too little may be insufficient for disintegration to occur and may thus alter the rate and extent of release of the active ingredient(s) from the dosage form. Thus, a sufficient amount of disintegrant that is neither too little nor too much to detrimentally alter the release of the active ingredient(s) should be used to form solid oral dosage forms of the disclosure. The amount of disintegrant used varies based upon the type of formulation and mode of administration, and is readily discernible to those of ordinary skill in the art. Typical pharmaceutical compositions comprise from about 0.5 to about 15 weight percent of disintegrant, preferably from about 1 to about 5 weight percent of disintegrant.

Disintegrants that can be used to form pharmaceutical compositions and dosage forms of the disclosure include, but are not limited to, agar, alginic acid, calcium carbonate, microcrystalline cellulose, croscarmellose sodium, crospovidone, polacrilin potassium, sodium starch glycolate, potato or tapioca starch, other starches, pre-gelatinized starch, clays, other algins, other celluloses, gums, and mixtures thereof.

Lubricants that can be used to form pharmaceutical compositions and dosage forms of the disclosure include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL® 200, manufactured by W. R. Grace Co. of Baltimore, Md.), a coagulated aerosol of synthetic silica (marketed by Degussa Co. of Plano, Tex.), CAB-O-SIL® (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

This disclosure further encompasses lactose-free pharmaceutical compositions and dosage forms, wherein such compositions preferably contain little, if any, lactose or other mono- or di-saccharides. As used herein, the term “lactose-free” means that the amount of lactose present, if any, is insufficient to substantially increase the degradation rate of an active ingredient.

Lactose-free compositions of the disclosure can comprise excipients which are well known in the art and are listed in the USP (XXI)/NF (XVI), which is incorporated herein by reference. In general, lactose-free compositions comprise a pharmaceutically acceptable salt of an embodiment of the present disclosure, a binder/filler, and a lubricant in pharmaceutically compatible and pharmaceutically acceptable amounts. Preferred lactose-free dosage forms comprise a pharmaceutically acceptable salt of the disclosed compounds, microcrystalline cellulose, pre-gelatinized starch, and magnesium stearate.

This disclosure further encompasses anhydrous pharmaceutical compositions and dosage forms comprising the disclosed compounds as active ingredients, since water can facilitate the degradation of some compounds. For example, the addition of water (e.g., 5%) is widely accepted in the pharmaceutical arts as a means of simulating long-term storage in order to determine characteristics such as shelf life or the stability of formulations over time. See, e.g., Jens T. Carstensen, Drug Stability: Principles & Practice, 379-80 (2nd ed., Marcel Dekker, NY, N.Y.: 1995). Water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms of the disclosure can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials) with or without desiccants, blister packs, and strip packs.

Controlled and Delayed Release Dosage Forms

Pharmaceutically acceptable salts of the disclosed compounds can be administered by controlled- or delayed-release means. Controlled-release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. Kim, Cherng-ju, Controlled Release Dosage Form Design, 2 (Technomic Publishing, Lancaster, Pa.: 2000).

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under-dosing a drug (i.e., going below the minimum therapeutic levels) as well as exceeding the toxicity level for the drug.

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the salts and compositions of the disclosure. Examples include, but are not limited to, those described in U.S. Pat. Nos. 3,845,770; 3,916,899; 3,536,809; 3,598,123; 4,008,719; 5674,533; 5,059,595; 5,591,767; 5,120,548; 5,073,543; 5,639,476; 5,354,556; 5,733,566; and 6,365,185 B1; each of which is incorporated herein by reference. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), multilayer coatings, microparticles, liposomes, or microspheres or a combination thereof to provide the desired release profile in varying proportions. Additionally, ion exchange materials can be used to prepare immobilized, adsorbed salt forms of the disclosed compounds and thus effect controlled delivery of the drug. Examples of specific anion exchangers include, but are not limited to, Duolite® A568 and Duolite® AP143 (Rohm & Haas, Spring House, Pa. USA).

One embodiment of the disclosure encompasses a unit dosage form that includes a pharmaceutically acceptable salt of the disclosed compounds (e.g., a sodium, potassium, or lithium salt), or a polymorph, solvate, hydrate, dehydrate, co-crystal, anhydrous, or amorphous form thereof, and one or more pharmaceutically acceptable excipients or diluents, wherein the pharmaceutical composition or dosage form is formulated for controlled-release. Specific dosage forms utilize an osmotic drug delivery system.

A particular and well-known osmotic drug delivery system is referred to as OROS® (Alza Corporation, Mountain View, Calif. USA). This technology can readily be adapted for the delivery of compounds and compositions of the disclosure. Various aspects of the technology are disclosed in U.S. Pat. Nos. 6,375,978 B; 6,368,626 B1; 6,342,249 B1; 6,333,050 B2; 6,287,295 B1; 6,283,953 B1; 6,270,787 B1; 6,245,357 B1; and 6,132,420; each of which is incorporated herein by reference. Specific adaptations of OROS® that can be used to administer compounds and compositions of the disclosure include, but are not limited to, the OROS® Push-Pull™, Delayed Push-Pull™, Multi-Layer Push-Pull™, and Push-Stick™ Systems, all of which are well known. See, e.g. worldwide website alza.com. Additional OROS® systems that can be used for the controlled oral delivery of compounds and compositions of the disclosure include OROS®-CT and L-OROS®; see, Delivery Times, vol. 11, issue II (Alza Corporation).

Conventional OROS® oral dosage forms are made by compressing a drug powder (e.g., a BH₄ salt) into a hard tablet, coating the tablet with cellulose derivatives to form a semi-permeable membrane, and then drilling an orifice in the coating (e.g., with a laser). Kim, Chemg-ju, Controlled Release Dosage Form Design, 231-238 (Technomic Publishing, Lancaster, Pa.: 2000). The advantage of such dosage forms is that the delivery rate of the drug is not influenced by physiological or experimental conditions. Even a drug with a pH-dependent solubility can be delivered at a constant rate regardless of the pH of the delivery medium. But because these advantages are provided by a build-up of osmotic pressure within the dosage form after administration, conventional OROS® drug delivery systems cannot be used to effectively delivery drugs with low water solubility. Because salts and complexes of this disclosure may be far more soluble in water than an embodiment of the present disclosure itself, they may be well suited for osmotic-based delivery to patients. This disclosure does, however, encompass the incorporation of embodiments of the present disclosure, and non-salt isomers and isomeric mixtures thereof, into OROS® dosage forms.

A specific dosage form of the compositions of the disclosure includes: a wall defining a cavity, the wall having an exit orifice formed or formable therein and at least a portion of the wall being semipermeable; an expandable layer located within the cavity remote from the exit orifice and in fluid communication with the semipermeable portion of the wall; a dry or substantially dry state drug layer located within the cavity adjacent the exit orifice and in direct or indirect contacting relationship with the expandable layer; and a flow-promoting layer interposed between the inner surface of the wall and at least the external surface of the drug layer located within the cavity, wherein the drug layer includes a salt of an embodiment of the present disclosure, or a polymorph, solvate, hydrate, dehydrate, co-crystal, anhydrous, or amorphous form thereof. See U.S. Pat. No. 6,368,626, the entirety of which is incorporated herein by reference.

Another specific dosage form of the disclosure includes: a wall defining a cavity, the wall having an exit orifice formed or formable therein and at least a portion of the wall being semipermeable; an expandable layer located within the cavity remote from the exit orifice and in fluid communication with the semipermeable portion of the wall; a drug layer located within the cavity adjacent the exit orifice and in direct or indirect contacting relationship with the expandable layer; the drug layer comprising a liquid, active agent formulation absorbed in porous particles, the porous particles being adapted to resist compaction forces sufficient to form a compacted drug layer without significant exudation of the liquid, active agent formulation, the dosage form optionally having a placebo layer between the exit orifice and the drug layer, wherein the active agent formulation comprises a salt of an embodiment of the present disclosure, or a polymorph, solvate, hydrate, dehydrate, co-crystal, anhydrous, or amorphous form thereof. See U.S. Pat. No. 6,342,249, the entirety of which is incorporated herein by reference.

Parenteral Dosage Forms

Parenteral dosage forms can be administered to patients by various routes, including, but not limited to, subcutaneous, intravenous (including bolus injection), intramuscular, and intraarterial. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared for administration of a patient, including, but not limited to, administration DUROS®-type dosage forms, and dose-dumping.

Suitable vehicles that can be used to provide parenteral dosage forms of the disclosure are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

Compounds that alter or modify the solubility of a pharmaceutically acceptable salt of an embodiment of the present disclosure disclosed herein can also be incorporated into the parenteral dosage forms of the disclosure, including conventional and controlled-release parenteral dosage forms.

Based on the foregoing studies, a dosage regime for BH₄ and other compounds or pharmaceutical compositions can be developed. In general, the starting dose of most Phase I clinical trials is based on preclinical testing, and is usually quite conservative. A standard measure of toxicity of a drug in preclinical testing is the percentage of animals (rodents) that die because of treatment. The dose at which 10% of the animals die is known as the LD₁₀, which has in the past often correlated with the maximal-tolerated dose (MTD) in humans, adjusted for body surface area. The adjustment for body surface area includes host factors such as, for example, surface area, weight, metabolism, tissue distribution, absorption rate, and excretion rate. Thus, the standard conservative starting dose is one tenth the murine LD₁₀, although it may be even lower if other species (i.e., dogs) were more sensitive to the drug. It is anticipated that a starting dose for BH₄ and other compounds or pharmaceutical compositions in Phase I clinical trials in humans will be determined in this manner. This dosing regimen is discussed in more detail in Freireich E J, et al., Cancer Chemother Rep 50:219-244, 1966, which is incorporated herein by reference.

Preferred unit dosage formulations are those containing a daily dose or unit, daily sub-dose, or an appropriate fraction thereof, of the administered ingredient. For example, approximately 5 milligrams per day of BH₄ can prevent diastolic dysfunction in mice. These results can be used to predict an approximate amount of the BH₄ to be administered to a human.

The approximation includes host factors such as surface area, weight, metabolism, tissue distribution, absorption rate, and excretion rate, for example. Therefore, approximately 15 to 20 grams per day of BH₄ should produce similar results in humans. As stated above, a therapeutically effective dose level will depend on many factors, as described above. In addition, it is well within the skill of the art to start doses of the composition at relatively low levels, and increase the dosage until the desired effect is achieved.

Topical, Transdermal and Mucosal Dosage Forms

Topical dosage forms of the disclosure include, but are not limited to, creams, lotions, ointments, gels, shampoos, sprays, aerosols, solutions, emulsions, and other forms known to one of skill in the art. See, e.g., Remington's Pharmaceutical Sciences, 18th ed., Mack Publishing, Easton, Pa. (1990); and Introduction to Pharmaceutical Dosage Forms, 4th ed., Lea & Febiger, Philadelphia, Pa. (1985). For non-sprayable topical dosage forms, viscous to semi-solid or solid forms comprising a carrier or one or more excipients compatible with topical application and having a dynamic viscosity preferably greater than water are typically employed. Suitable formulations include, without limitation, solutions, suspensions, emulsions, creams, ointments, powders, liniments, salves, and the like, which are, if desired, sterilized or mixed with auxiliary agents (e.g., preservatives, stabilizers, wetting agents, buffers, or salts) for influencing various properties, such as, for example, osmotic pressure. Other suitable topical dosage forms include sprayable aerosol preparations wherein the active ingredient, preferably in combination with a solid or liquid inert carrier, is packaged in a mixture with a pressurized volatile (e.g., a gaseous propellant, such as freon), or in a squeeze bottle. Moisturizers or humectants can also be added to pharmaceutical compositions and dosage forms if desired. Examples of such additional ingredients are well known in the art. See, e.g., Remington's Pharmaceutical Sciences, 18.sup.th Ed., Mack Publishing, Easton, Pa. (1990).

Transdermal and mucosal dosage forms of the compositions of the disclosure include, but are not limited to, ophthalmic solutions, patches, sprays, aerosols, creams, lotions, suppositories, ointments, gels, solutions, emulsions, suspensions, or other forms known to one of skill in the art. See, e.g., Remington's Pharmaceutical Sciences, 18th Ed., Mack Publishing, Easton, Pa. (1990); and Introduction to Pharmaceutical Dosage Forms, 4th Ed., Lea & Febiger, Philadelphia, Pa. (1985). Dosage forms suitable for treating mucosal tissues within the oral cavity can be formulated as mouthwashes, as oral gels, or as buccal patches. Additional transdermal dosage forms include “reservoir type” or “matrix type” patches, which can be applied to the skin and worn for a specific period of time to permit the penetration of a desired amount of active ingredient.

Examples of transdermal dosage forms and methods of administration that can be used to administer the active ingredient(s) of the disclosure include, but are not limited to, those disclosed in U.S. Pat. Nos. 4,624,665; 4,655,767; 4,687,481; 4,797,284; 4,810,499; 4,834,978; 4,877,618; 4,880,633; 4,917,895; 4,927,687; 4,956,171; 5,035,894; 5,091,186; 5,163,899; 5,232,702; 5,234,690; 5,273,755; 5,273,756; 5,308,625; 5,356,632; 5,358,715; 5,372,579; 5,421,816; 5,466;465; 5,494,680; 5,505,958; 5,554,381; 5,560,922; 5,585,111; 5,656,285; 5,667,798; 5,698,217; 5,741,511; 5,747,783; 5,770,219; 5,814,599; 5,817,332; 5,833,647; 5,879,322; and 5,906,830, each of which are incorporated herein by reference in their entirety.

Suitable excipients (e.g., carriers and diluents) and other materials that can be used to provide transdermal and mucosal dosage forms encompassed by this disclosure are well known to those skilled in the pharmaceutical arts, and depend on the particular tissue or organ to which a given pharmaceutical composition or dosage form will be applied. With that fact in mind, typical excipients include, but are not limited to water, acetone, ethanol, ethylene glycol, propylene glycol, butane-1,3-diol, isopropyl myristate, isopropyl palmitate, mineral oil, and mixtures thereof, to form dosage forms that are non-toxic and pharmaceutically acceptable.

Depending on the specific tissue to be treated, additional components may be used prior to, in conjunction with, or subsequent to treatment with pharmaceutically acceptable salts of an embodiment of the present disclosure. For example, penetration enhancers can be used to assist in delivering the active ingredients to or across the tissue. Suitable penetration enhancers include, but are not limited to: acetone; various alcohols such as ethanol, oleyl, an tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as TWEEN 80 (polysorbate 80) and SPAN 60 (sorbitan monostearate).

The pH of a pharmaceutical composition or dosage form, or of the tissue to which the pharmaceutical composition or dosage form is applied, may also be adjusted to improve delivery of the active ingredient(s). Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of the active ingredient(s) so as to improve delivery. In this regard, stearates can serve as a lipid vehicle for the formulation, as an emulsifying agent or surfactant, and as a delivery-enhancing or penetration-enhancing agent. Different hydrates, dehydrates, co-crystals, solvates, polymorphs, anhydrous, or amorphous forms of the pharmaceutically acceptable salt of an embodiment of the present disclosure can be used to further adjust the properties of the resulting composition.

Kits

A typical kit includes a unit dosage form of a composition (e.g., BH₄) or a pharmaceutically acceptable salt of an embodiment of the present disclosure. In particular, the composition or the pharmaceutically acceptable salt of an embodiment of the present disclosure is the sodium, lithium, or potassium salt, or a polymorph, solvate, hydrate, dehydrate, co-crystal, anhydrous, or amorphous form thereof. A kit may further include a device that can be used to administer the active ingredient. Examples of such devices include, but are not limited to, syringes, drip bags, patches, and inhalers.

Kits of the disclosure can further include vehicles or pharmaceutically acceptable vehicles that can be used to administer one or more active ingredients (e.g., BH₄). For example, if an active ingredient is provided in a solid form that must be reconstituted for parenteral administration, the kit can include a sealed container of a suitable vehicle in which the active ingredient can be dissolved to form a particulate-free sterile solution that is suitable for parenteral administration. Examples of pharmaceutically acceptable vehicles include, but are not limited to: water for injection USP; aqueous vehicles such as, but not limited to, sodium chloride injection, Ringer's injection, dextrose injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

Animal Model

The present disclosure also provides an animal in which DD has been established and a method of generating such an animal. The animal of the present disclosure is suitable for use as a model for studying DD. In particular, a mouse model was developed for studying DD using mice with DOCA-salt induced hypertension. The mice have an intact systolic function, the mice are characterized by rapid onset of diastolic dysfunction that is completely reversible, the mice are characterized by the absence of LV hypertrophy, and the mice are characterized by the absence of aostic or mitral regurgetation.

DOCA is administered in a sufficient amount to cause or generate hypertension and DD in the animal or to cause or generate hypertension and DD symptoms in the animal or a fetus. The sufficient amount typically varies between animals and will depend on a number of factors.

DOCA may be administered to the animals by methods well known in the art. DOCA can be administered orally, for example as tablets, troches, lozenges, aqueous or oily suspensions, dispersible powders or granules. DOCA may also be administered parenterally, either subcutaneously, intravenously, intramuscularly, intrasternally, transdermally or by infusion techniques.

The animal is non-human. The non-human animal is typically of a species commonly used in biomedical research, for example a mammal, and is preferably a laboratory strain. Suitable animals include non-human primates, dogs, cats, sheep and rodents. It is preferred that the animal is a rodent, particularly a mouse, rat, guinea pig, ferret, gerbil or hamster. Most preferably the animal is a mouse.

Typically a suitable non-human animal is a so-called “knock-out animal”. The term “knock-out animal” is well known to those skilled in the art. A knock-out animal can be produced according to any suitable method.

Additional details regarding the DOCA mouse are described in the Examples.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include ±1%, ±2%, ±3%, ±4%, ±5%, ±6%, ±7%, ±8%, ±9%, or ±10%, or more of the numerical value(s) being modified. In addition, the phrase “about ‘x’ to ‘y’” includes “about ‘x’ to about ‘y’”.

The above discussion is meant to be illustrative of the principles and various embodiments of the present disclosure. Numerous variations and modifications will become apparent to those skilled in the art once the above disclosure is fully appreciated. It is intended that the following claims be interpreted to embrace all such variations and modifications.

### EXAMPLES

### Example 1

Diastolic dysfunction is a growing health problem, and there is currently no proven therapy.

We established an animal model of isolated diastolic dysfunction using mice with deoxycorticosterone acetate (DOCA)-induced hypertension. The development of diastolic dysfunction was monitored by echocardiography using two relatively load-independent indices of diastolic function: V_{P}—left ventricular (LV) inflow propagation velocity and E′—early diastolic LV longitudinal velocity by pulsed-wave tissue Doppler imaging. Invasive hemodynamic measurements were also performed in these mice to confirm the presence of diastolic dysfunction. Tissue biopterin content was measured using HPLC. Dimeric and monomeric forms of endothelial NOS (eNOS) in the heart were detected by Western blot.

Compared to the sham-operated animals, the DOCA-salt treated mice were mildly hypertensive (systolic blood pressure 123±3 vs. 97±3 mmHg, p<0.05), had decreased V_{P} (23±1 vs. 45±1 cm/s, p<0.05) and E′ (2.9±0.2 vs. 5.4±0.3 cm/s, p<0.05), a prolonged LV relaxation time constant, tau (14.4±0.8 vs. 8.7±1.0 ms, p<0.05), a lower relaxation index, dP/dtₘᵢₙ/P (100±6 vs. 130±7 s⁻¹, p<0.05), and an elevated LV end-diastolic pressure (10.3±0.8 vs. 4.9±1.6 mmHg, p<0.05). The LV contractility index, dP/dtₘₐₓ/P, was not different between the 2 groups. There was no LV hypertrophy in the DOCA mice. The DOCA mice hearts showed increased levels of oxidized biopterins and decreased ratios of dimeric to monomeric eNOS consistent with a cardiac oxidative state. Feeding BH₄ (5 mg/day) to DOCA mice improved cardiac BH₄ store, increased the ratio of dimeric to monomeric eNOS, and prevented the development of diastolic dysfunction.

The development of diastolic dysfunction in the DOCA hypertensive mice was associated with increased biopterin oxidation. BH₄ feeding augmented cardiac reduced biopterin levels and prevented diastolic dysfunction.

Methods

Materials.

BH₄ [(6R)-5,6,7,8-tetrahydro-L-biopterin dihydrochloride] was purchased from AXXORA (San Diego, Calif.). The compound is stable for several years when stored at −20° C. All other reagents were purchased from Sigma-Aldrich (St. Louis, Mo.).

Dietary Supplementation with BH₄.

BH₄ was compressed into standard rodent chow pellets without addition of water or heating to prevent oxidation of the compound (Bio-Serv, Frenchtown, N.J.). The concentration of BH₄ in the pellets (1 mg/g) was designed to provide a daily dose of 5 mg, based on an average mouse dietary intake of 5 g per day. Pellets were stored in a sealed bag at −20° C. to retard spontaneous oxidation.

Blood Pressure and Heart Rate Measurements.

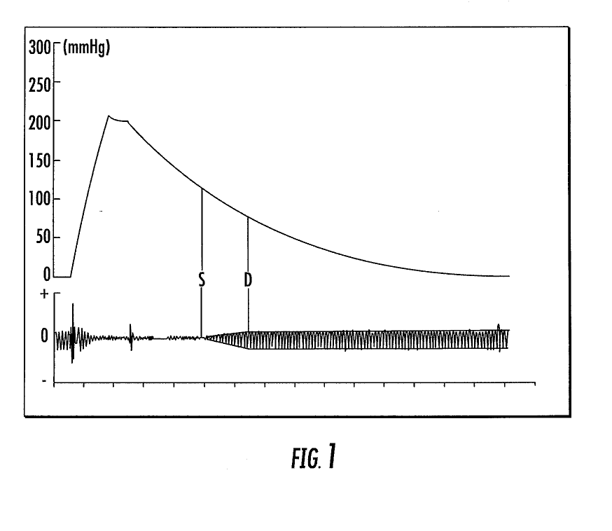

Resting blood pressure (BP) and heart rate (HR) were measured by tail-cuff plethysmography using the 8-Channel Non-Invasive Blood Pressure Monitor (Columbus Instruments, Columbus, Ohio). All animals were acclimated to the NIBP-8 environment before any actual measurements are undertaken. The NIBP-8 system acquires the tail artery pulse and corresponding pressure signals through a pressurized sensor cuff during the transition between inflation and deflation of an occlusion cuff. A representative plethysmographic tracing from a conscious normal mouse is shown in FIG. 1. In this example, the HR is 562 bpm and the systolic and diastolic BP is 114 mmHg and 78 mmHg, respectively. Our group has shown that tail cuff plethysmography is similar to invasive, telemetric monitoring under this experimental condition.

Echocardiography.

The mouse was lightly anesthetized with isoflurane. Isoflurane (1%) was delivered through a face mask at a rate of 5 L/min. The mouse was kept warm on a heating pad. The body temperature was continuously monitored using a rectal thermometer probe and maintained between 36 and 37° C. by adjusting the distance of a ceramic heating lamp. Under these conditions, the animal's heart rate could be maintained above 400 beats per minute. Transthoracic echocardiography was performed using a SONOS 5500 ultrasound unit (Philips Medical Systems, Bothell, Wash.) equipped with a 15-MHz linear-array transducer and a 12-MHz phase-array transducer. The high-frequency 15-MHz linear-array transducer was used to acquire 2-dimensional and M-mode images from the parasternal long-axis view and the LV short-axis view at the mid-papillary level for the measurement of LV chamber dimensions and wall thickness. The 12-MHz phase-array transducer was used to acquire pulsed-wave Doppler, continuous-wave Doppler, color-flow Doppler, color M-mode Doppler, and tissue Doppler images from the apical 4-chamber view for the assessment of LV diastolic function and valvular integrity. LV diastolic function was evaluated by the conventional pulsed-wave Doppler recording of mitral inflow velocities (E and A) and two of the newer, relatively load-independent, echocardiographic indices of diastolic function: LV inflow propagation velocity (V_{P}) by color M-mode Doppler, and mitral annulus longitudinal velocities (E′ and A′) by pulsed-wave tissue Doppler imaging (TDI). These two newer echocardiographic indices of diastolic function have been shown by many investigators to be relatively insensitive to preload and heart rate. We have also observed in our laboratory that transient occlusion of inferior vena cava (IVC) had little effect on V_{P} or mitral annulus longitudinal velocities, confirming the preload-insensitive attribute of these two echocardiographic indices of diastolic function in mice. Also, we have found a strong correlation between echocardiographic and invasive hemodynamic measures under our experimental conditions, suggesting that echocardiography is a valuable complement to the more traditional invasive methods. Echocardiograms were acquired at baseline and at 14-day post DOCA-pellet implantation.

Hemodynamic Measurements In Vivo.

Cardiac hemodynamics were measured after the final echocardiographic examination. Mice were anesthetized with 1% isoflurane and ventilated. Body temperature was monitored using a rectal thermometer probe and maintained between 36 and 37° C. using a heating pad and a heating lamp. A 1.4-Fr Mikro-Tip catheter pressure transducer (Millar Instruments, Houston, Tex.) was inserted into the right common carotid artery and advanced through the aortic valve into the LV for continuous LV pressure measurements. The catheter was calibrated using an external analog manometer. Data were acquired at a sampling rate of 1,000 Hz using a PowerLab system and analyzed using Chart 5 software (ADInstruments, Colorado Springs, Colo.). Each animal was allowed to stabilize for at least 10 min or until stable HR, LV systolic pressure, and maximal rate of pressure development (dP/dtₘₐₓ) were observed. Baseline values of HR and LV pressures were then recorded for subsequent analysis.

Measurements of Cardiac Biopterin Content.

Cardiac biopterin content was measured using HPLC analysis and a differential oxidation method, as described by Antonozzi et al. (1988). A known amount of ventricular myocardial tissue was homogenized in a 0.1 N phosphate buffer at a pH of either 12.0 or 2.0. Pterins at the two pHs were differentially oxidized by exposure to 1% iodine/2% potassium iodide. After the differential oxidation, the particulate material was removed by centrifugation at 3,000 g for 30 min. Supernatants were then passed over a Dowex 50 column to concentrate the pterins and to eliminate other fluorescent molecules. HPLC was performed using a C₁₈ column (5×250 mm, 5 μm) and a mobile phase of 5% methanol and 95% water at a flow rate of 1 ml/min. Peaks were detected using a fluorescence detector with authentic biopterin as the standard. The fluorescence detector was set at 350 nm for excitation and 450 nm for emission. The amount of BH₄ was determined from the difference between total biopterins (BH₄ plus BH₂ plus biopterin) and alkaline-stable oxidized biopterins (BH₂ plus biopterin). Biopterin levels were expressed as picomoles per mg protein. Protein concentration was measured by the method of Lowry et al (1951) with bovine serum albumin as the standard. An example of the HPLC tracings obtained from a normal mouse heart is shown in FIG. 5.

Detection of Cardiac eNOS Expression.

SDS-resistant eNOS dimers and monomers were assayed using low-temperature SDS-PAGE under reducing or nonreducing conditions, as described by Zou et al. (2002). Tissue samples were added to 5-fold Laemmli buffer (0.32 mol/l Tris-HCl, pH 6.8, 0.5 mol/l glycine, 10% SDS, 50% glycerol, and 0.03% bromophenol blue) in nonreducing gel (without 2-mercaptoethanol) to identify dimer dissociation due to reduced disulfide bridges. To provide fully denatured control lanes, samples were boiled for 15 min prior to loading. Electrophoresis was performed using Tris glycine 6% gels, and gels and buffers were maintained in an ice bath at 4° C.

Data Analysis.

Data are presented as the mean ±one standard error. Differences in continuous variables between two groups are assessed by Student's t test for parametric data and by χ² analysis for categorical data. Comparison among multiple groups is performed by one-way ANOVA and a post-hoc test when significance is indicated. A p value <0.05 is considered statistically significant.

Results

Evidence of Diastolic Dysfunction by Echocardiographic Measures.

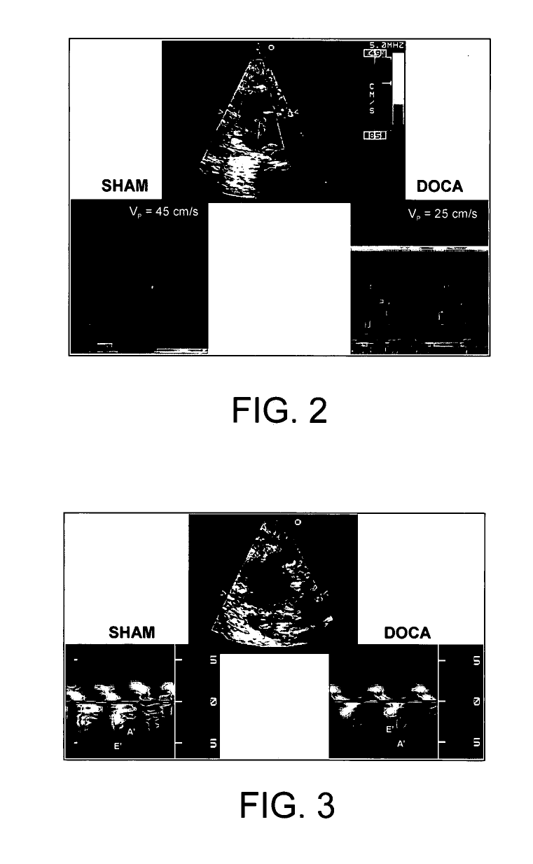

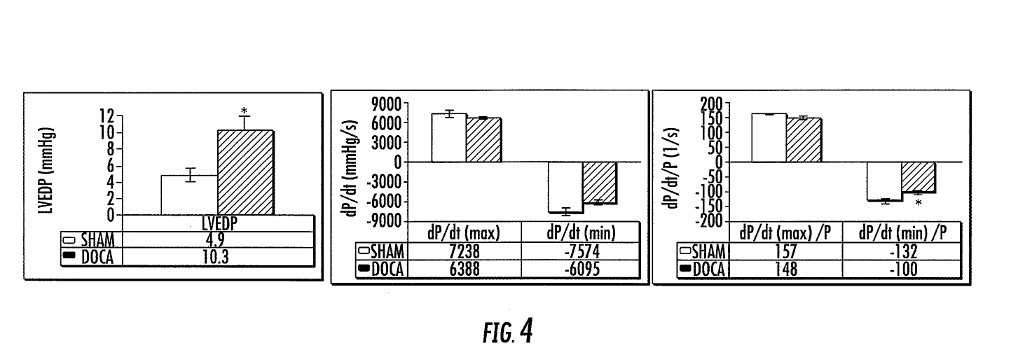

Compared to sham-operated animals, the DOCA mice were mildly hypertensive (systolic blood pressure 123±3 vs. 97±3 mmHg, p<0.05). The DOCA mice also exhibited signs of diastolic dysfunction, as evidenced by a blunted LV inflow propagation velocity, V_{P} (23±1 vs. 45±1 cm/sec, p<0.05; FIG. 2), a reduced early diastolic LV longitudinal velocity, E′ (2.9±0.2 vs. 5.4±0.3 cm/sec, p<0.05; FIG. 3), and an abnormal E′/A′ ratio of <1 (Table 1, Example 1). Dietary supplementation with BH₄, beginning the day after nephrectomy, prevented the diastolic abnormalities seen in the DOCA mice. There was no evidence of valvular insufficiency or LV hypertrophy in the DOCA mice.

TABLE 1
Example 1

| | SBP | DBP | HR | Vₚ | E′ |
|---|---|---|---|---|---|
| | (mmHg) | (mmHg) | (bpm) | (cm/s) | (cm/s) |
| SHAM | 97 ± 3 | 74 ± 3 | 557 ± 10 | 45 ± 1 | 5.4 ± 0.3 |
| DOCA | 123 ± 3* | 94 ± 3* | 548 ± 15 | 23 ± 1* | 2.9 ± 0.2* |
| DOCA + | 98 ± 6^{§} | 75 ± 4^{§} | 576 ± 19 | 40 ± 1^{§} | 4.8 ± 0.3^{§} |
| BH4 | | | | | |

Values are mean ± SEM, n = 7 for each group.
*p < .05 vs. SHAM;
^{§}p < .05 vs. DOCA.

Evidence of Diastolic Dysfunction by Invasive Hemodynamic Measures.

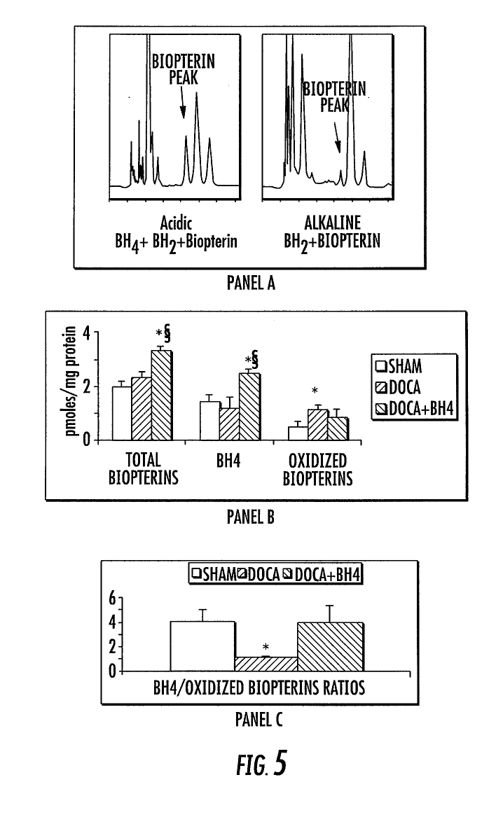

As shown in FIG. 4, the DOCA mice had a significantly elevated LVEDP compared to the sham-operated mice (10.3±0.8 vs. 4.9±1.6 mmHg, p<0.05). The dP/dtₘₐₓ/P (dP/dtₘₐₓ corrected by corresponding LV pressure), an index of LV systolic function, was not statistically different between the DOCA mice and the sham-operated mice (148±157±s⁻¹, p>0.05). In contrast, the dP/dtₘᵢₙ/P (dP/dtₘᵢₙ corrected by corresponding LV pressure), an index of LV diastolic function, was significantly decreased in the DOCA mice compared to the sham-operated mice (100±6 vs. 130±7 s⁻¹, p<0.05). The DOCA mice also had a significantly prolonged LV relaxation time constants (τ) compared to the sham-operated mice (14.4±0.8 vs. 8.7±1.0 ms, p<0.05). The findings are consistent with diastolic dysfunction with intact systolic function.

Cardiac BH₄ to Oxidized Biopterins Ratio is Decreased in DOCA-Salt Hypertensive Mice.

The hearts of DOCA mice were found to have an elevated level of oxidized biopterins (BH₂+biopterin—approximately two times the amount found in the sham-operated mice (1.12±0.15 vs. 0.52±0.19 μmol/mg, p<0.05; FIG. 5, Panel B). The ratio of cardiac BH₄ to oxidized biopterins was approximately 4-fold lower in the DOCA mice than in the sham-operated mice (1.1±0.1 vs. 4.1±0.9, p<0.05; FIG. 5, Panel C). The total cardiac biopterins levels (BH₄+BH₂+biopterin) were not significantly different between the DOCA group and the sham-operated group. These data are strong evidence for a cardiac oxidative state in the DOCA-salt hypertensive mice. Feeding BH₄ to DOCA mice significantly augmented their total cardiac biopterins levels (3.30±0.16 vs. 2.32±0.40 μmol/mg, p<0.05) and cardiac BH₄ levels (2.48±0.21 vs. 1.21±0.26 μmol/mg, p<0.05). BH₄ feeding to DOCA mice also restored the ratio of cardiac BH₄ to oxidized biopterins to normal.

Decreased Presence of Dimeric eNOS in the Heart of DOCA-Salt Hypertensive Mice.

All three isoforms of NOS are dimeric enzymes comprised of two identical subunits, and NOS is catalytically active only in dimeric form (Zou et al., 2002). No eNOS bands were detectable in the heart of eNOS^{-/-} mouse. Cultured endothelial cell samples were included as positive controls. Upon boiling, all dimeric eNOS proteins denatured to the monomeric form, forming a single band as shown. Compared to the sham-operated mice, the DOCA mice had a decreased presence of dimeric eNOS and an increased presence of monomeric eNOS. These DOCA-salt induced changes were prevented by BH₄ feeding. Total eNOS—the combined amounts of dimers and monomers—were comparable among the 3 groups of animals.

Discussion

There are three major findings in the present study. First, DOCA-salt treated mice developed signs of diastolic dysfunction within 2 weeks of hypertension induction. Second, the development of diastolic dysfunction in the DOCA-salt hypertensive mice was accompanied by increased cardiac biopterin oxidation and a lowering of the dimeric/monomeric eNOS ratio, consistent with a cardiac oxidative state. Third, BH₄ feeding restored cardiac reduced biopterin store and diastolic dysfunction.

To our knowledge, this is the first study demonstrating the presence of diastolic dysfunction in the DOCA-salt hypertensive mouse model. This new mouse model of diastolic dysfunction is characterized by several important features that allow for relatively direct interpretations of experimental results, including: 1) intact systolic function, 2) rapid onset of diastolic dysfunction that is completely reversible, 3) absence of LV hypertrophy, and 4) absence of aortic or mitral regurgitation. In the present study, we have also demonstrated that tissue Doppler imaging (TDI) and color M-mode Doppler echocardiography are very valuable tools for the assessment of diastolic function in mice. In addition to invasive hemodynamic approaches available in our laboratory, these non-invasive techniques allow us the ability to perform serial examinations on the same animal, so that the onset, progression, and regression of diastolic dysfunction can be closely monitored. TDI is a relatively new echocardiographic technique that is increasingly gaining popularity as a diagnostic tool for diastolic dysfunction. It has been shown to be relatively insensitive to preload, and is therefore particularly helpful in differentiating normal from pseudonormal filling pattern. TDI employs the Doppler principle to measure the velocity of myocardial segments and other cardiac structures. Impairment of longitudinal cardiac motion is a sensitive marker of early myocardial dysfunction and ischemia. TDI allows quantitative measurement of long-axis ventricular function. Mitral annulus velocity in diastole is reflective of changes in velocity for the LV long axis. In normal hearts, the long axis and circumferential motion is approximately the same. By recording mitral annulus motion from the apex, the effect of myocardial translation is minimized. A typical spectral pattern will demonstrate a single systolic velocity toward the LV centroid (Sm), and two signals away from the centroid during early and late diastole (FIG. 3). With abnormal active relaxation, mitral annulus velocity during early diastole (E′) is decreased while mitral annulus velocity during late diastole (A′) is increased, resulting in a lowered E′/A′ ratio. In restrictive cardiomyopathy, both the E′ and A′ are severely blunted. In contrast, the mitral annulus velocity is preserved in constrictive pericarditis. TDI has recently been validated as a reliable tool in the evaluation of DD in mice. LV inflow propagation velocity (V_{P}) by color M-mode Doppler is another preload insensitive index of LV relaxation. It differs from conventional pulsed-wave Doppler in that it allows the acquisition of spatial information, in addition to velocity and time information. It has been shown to correlate well with the time constant of isovolumic relaxation (τ), both in animals and humans. In anesthetized dogs, V_{P} has proved to be independent of left atrial pressure and heart rate. V_{P} has also been shown to reflect changes in myocardial relaxation in mice with genetically altered levels of phospholamban.

There is accumulating evidence that heart failure is associated with increased oxidative stress and that increased oxidative stress may contribute to the progression of heart failure. Direct evidence that free radical production and oxidative stress play a key role in the triggering and progression of heart failure came from experiments studying cardiomyopathy induced by iron overload. In both subacute and chronically iron-overloaded hearts, there was clear evidence of increased oxidative damage, as shown by marked increases in various lipid peroxidation products (aldehydes) and depletion of GSH (and GSH+GSSG) levels. Iron-overloaded mice had markedly elevated levels of unsaturated (malondialdehyde and 4-hydroxynonenal) and saturated (hexanal) aldehydes in the heart and plasma. These aldehyde products are generated by free radical-induced lipid peroxidation and participate in cytotoxic reactions, leading to cellular dysfunction.

Cardiovascular risk factors such as hypertension, hypercholesterolemia, diabetes mellitus, or chronic smoking stimulate the production of ROS in the vascular wall. Nicotinamide adenine dinucleotide phosphate (NADPH) oxidases represent major sources of this ROS and have been found upregulated and activated in animal models of hypertension, diabetes, and sedentary lifestyle and in patients with cardiovascular risk factors. Superoxide reacts avidly with vascular NO to form peroxynitrite (ONOO—). The cofactor BH₄ is highly sensitive to oxidation by ONOO—.

As discussed above, BH₄ supplementation has been shown to confer a variety of cardiovascular benefits in preclinical studies. Our present study further indicates that BH₄ supplementation may have a therapeutic potential for diastolic dysfunction and diastolic heart failure. Our findings are of particular importance because diastolic dysfunction and diastolic heart failure represent a tremendous public health burden in the U.S. and yet there is currently no proven therapy available for this condition. BH₄ is an FDA-approved drug used clinically to treat some forms of phenylketonuria. Its safety in humans is well established. Recently, a pilot study from our institution demonstrated that BH₄ is a safe and effective antihypertensive agent in patients with poorly controlled hypertension. Large scale clinical studies are warranted to define the full therapeutic potential of BH₄ in cardiovascular medicine.

### Example 2

### Measuring Cardiac Oxidative Stress

Our interest in oxidative stress as a cause of diastolic dysfunction arose from experiments that we published previously concerning the role of oxidative stress in atrial fibrillation (AF). Because the atrial endocardium shares many characteristics with the arterial endothelium, we hypothesized that it may demonstrate alterations in redox active proteins in response to changes in shear stress that could increase atrial oxidative stress during AF. To test this hypothesis, we studied a pig model of AF. A specially designed pacemaker was inserted into the right atrium (RA), and the atrium paced at a rate of 600 bpm to induce AF. The atrioventricular (AV) node was abolished using radiofrequency ablation, and a pacemaker was inserted into the right ventricle (RV) to maintain ventricular rates at 100 bpm. Control pigs had AV nodal ablation and subsequent right-sided AV sequential pacing at 100 bpm, such that the ventricular rate was identical in both sets of animals. ECG confirmed AF at the end of the procedure and before sacrifice. AF animals showed no evidence of congestive heart failure. After one week, animals were euthanized with sodium pentobarbital intravenously, and the hearts rapidly excised. The hearts were rinsed and dissected in Krebs-HEPES buffer, and tissue was either studied immediately or quick frozen in liquid nitrogen for subsequent enzymatic assays and Western analysis.

Custom built NO electrodes were fabricated with coated carbon fibers. The electrodes were calibrated with serial dilutions of a saturated, degassed NO solution. During voltammetry, the electrode showed a characteristic peak consistent with the oxidation potential of NO. The calibration curve was linear with respect to NO with a detection limit around 10 nM. We have published six manuscripts on the design and use of this particular NO electrode variant, establishing its validity and differentiating it from a less reliable commercially available substitute.

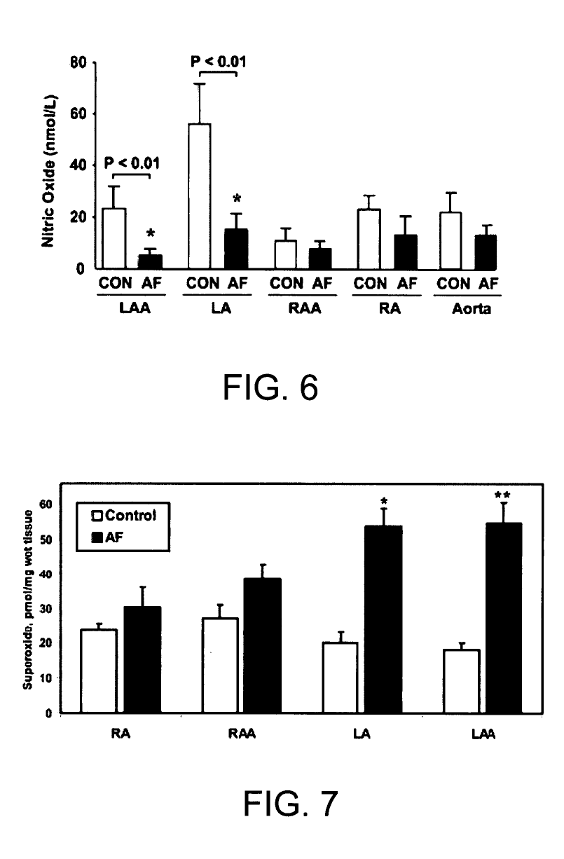

Isolated tissue was placed in an organ bath with the endocardium facing upward. At 37° C., NO concentration was measured under basal conditions and after stimulation with the calcium ionophore, A23187 (1 μmol/L). Interestingly, basal NO concentration was three fold higher in the LA than any of the other tissues studied. AF for one week decreased NO concentration by almost one-fourth (15±6 vs. 56±16, p<0.01, FIG. 6). Also, AF dramatically decreased stimulated LA NO release (31±12 nmol/L vs. 107±34 nmol/L for control left atria, p<0.01). The effects of AF on NO concentration were comparable in the left atrial appendage (LAA). AF did not cause a significant change in basal or stimulated NO concentration in the ascending Aorta (Ao).

NOS expression from AF and control animals was quantified with Western blot analysis using a monoclonal antibody against eNOS. Surprisingly, there was no significant difference in NOS expression between control and AF animals in the LM, despite the observation that NO levels were decreased 3 fold in the AF group. One possible explanation for the reduction in NO is increased oxidative degradation by O₂. We investigated this possibility in the experiments described below.

After a week of AF induced by rapid atrial pacing in pigs, O₂ production from acutely isolated heart tissue was measured by two independent techniques, electron spin resonance (ESR) and SOD-inhibitable cytochrome C reduction assays. Compared to control animals with equivalent ventricular heart rates, basal O₂ production was increased 2.7 (p<0.01) and 3.0 fold (p<0.02) in the LA and LAA, respectively (FIG. 7). A similar 3.0 fold (p<0.01) increase in LAA O₂ production was observed using a cytochrome C reduction assay. The increases could not be explained by changes in atrial total SOD activity.

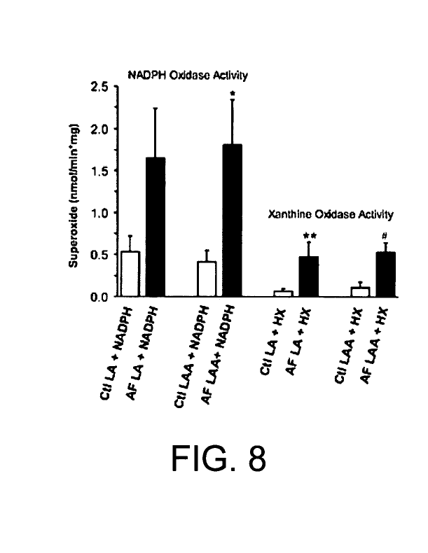

NOX appeared to play a central role in this myocardial oxidative stress. The NOX inhibitor, apocynin (100 μg/mL), reduced LAA O₂ production by 91%, suggesting a role of the NOX. To investigate this further, LAA NOX activities were compared directly using membrane preparations from AF and control pigs. There was a 4.4 fold increase in NOX activity in the LM of pigs with AF (FIG. 8, p=0.02). The NOX activity was 0.4±0.1 in controls compared to 1.8±0.5 nmol O₂/mg tissue*min in AF pigs. The LA showed a similar trend toward increased NOX activity (p=0.06).

Because xanthine oxidase is another source of O₂ which can be activated concomitantly with the NOX, we also investigated changes in xanthine oxidase activity caused by AF. Although the overall O₂ production attributable to this system was lower than that of the NOX, incubation of LAA with oxypurinol reduced O₂ production by 85%. Furthermore, LM xanthine oxidase activity also showed a 4.4 fold increase with AF (p=0.01), rising from 0.1±0.1 in control to 0.5±0.1 nmol O₂/mg tissue*min in AF pigs. In the case of xanthine oxidase, the differences between activities in the control and AF groups in the LA were also statistically significant (p=0.04).

In summary, we demonstrated that AF was associated with regionally correlated decreases in myocardial NO and increases in O₂ production. The increased O₂ production was at least in part the result of increased NOX and xanthine oxidase activities. Increased NOX activity could be explained by an increase in active Rac1, a required cofactor.

A Hypertension Model of LV Diastolic Dysfunction:

We have developed an animal model of diastolic dysfunction using mice with DOCA-salt induced hypertension. Our group has considerable experience using this model for the study of vascular diseases and has previously shown that the mice show increased NOX-dependent oxidative stress. This application will explore the effects of these changes on the heart. The cardiac phenotype of the animal model is characterized by several important features, including: 1) rapid onset of diastolic dysfunction that is completely reversible, 2) intact systolic function, 3) absence of left ventricular hypertrophy, and 4) absence of aortic or mitral regurgitation. These features allow for an assessment of diastolic dysfunction with relatively few confounding variables.

Hypertension was induced in 7-week-old male C57BL/6 mice by unilateral nephrectomy, subcutaneous implantation of a slow-release DOCA pellet (15 mg released over a 21-day period), and substituting drinking water with 1% saline. Blood pressure and heart rate were monitored by tail-cuff plethysmography in conscious, acclimated mice.

TABLE 1
Example 2
Evidence of reversible diastolic dysfunction in DOCA mice.

| | | Systolic | Diastolic | heart | | | |
|---|---|---|---|---|---|---|---|
| | n | blood pressure | blood pressure | rate | Vₚ | E′ | E′/A′ |
| SHAM | 7 | 97 ± 3 | 74 ± 3 | 557 ± 10 | 45 ± 1 | 5.4 ± 0.3 | 1.7 ± 0.2 |
| DOCA | 7 | 123 ± 3* | 94 ± 3* | 548 ± 15 | 23 ± 1* | 2.9 ± 0.2* | 0.7 ± 0.1* |
| DOCA + BH₄ | 7 | 98 ± 6^{§} | 75 ± 4^{§} | 576 ± 19 | 40 ± 1^{§} | 4.8 ± 0.3^{§} | 1.7 ± 0.2^{§} |

Values are mean ± SEM.
*p < 0.05 vs. SHAM group;
^{§}p < 0.05 vs. DOCA group.

We confirmed tail-cuff measurements with ambulatory blood pressure telemetry and invasive hemodynamics. As seen by others, these three measures showed identical trends. Two weeks after DOCA pellet implantation and saline feeding, the treated mice were found to have a mildly elevated blood pressure (Table 1, Example 2). Transthoracic echocardiography revealed an abnormally low LV inflow propagation velocity and an abnormally low E′/A′ ratio consistent with diastolic dysfunction. The systolic function was normal, and there was no evidence of left ventricular hypertrophy by M-mode echocardiography. To evaluate whether the observed changes in diastolic function could be reversed, we discontinued saline feeding and followed the diastolic function of these animals with serial echocardiography. We observed complete resolution of all diastolic abnormalities 3 weeks after cessation of saline feeding (or 2 weeks past the 21-day DOCA release period). The observation suggests that no permanent structural remodeling had occurred in the heart of these animals.

Hypertensive Mice Show Diastolic Dysfunction by Echocardiography:

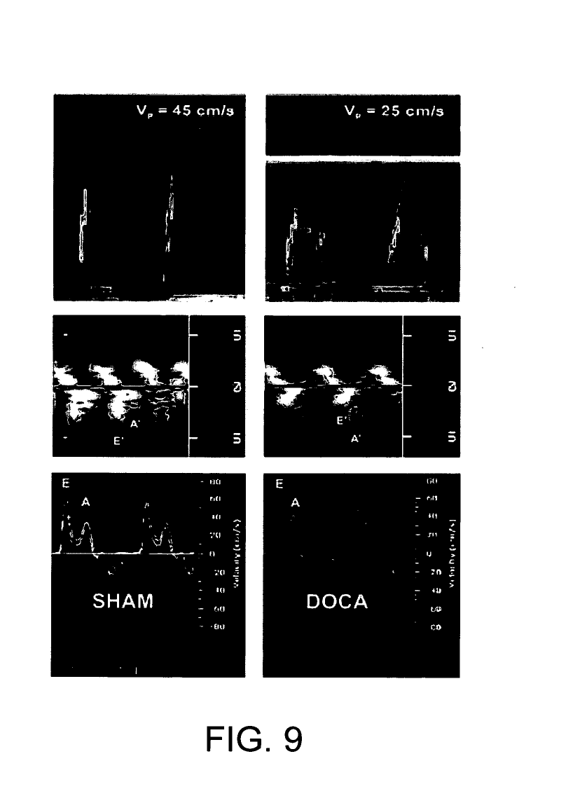

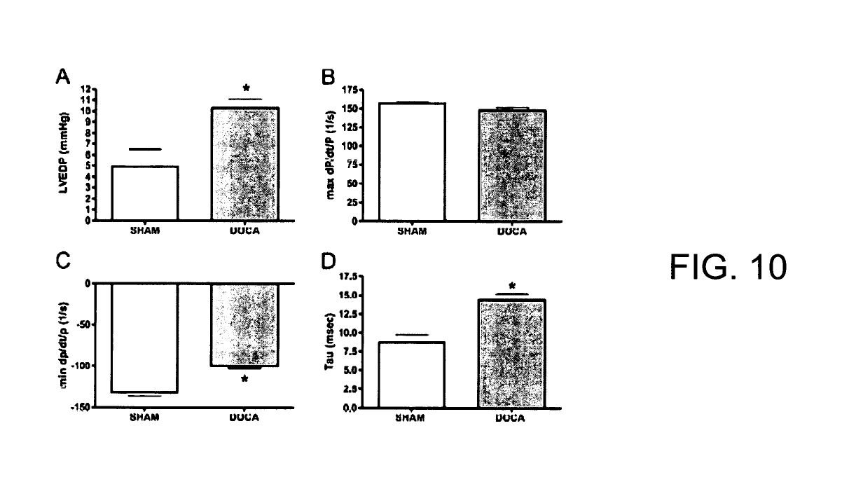

Echocardiography allows the ability to perform serial examinations on the same animal, so that the onset, progression, and regression of diastolic dysfunction can be closely monitored under various experimental conditions. These measures have correlated well with and will complement the invasive measures. In the future, echocardiographic measures will be done under conditions of variable load to better resolve load-independent parameters. For diastolic dysfunction assessment, the mice were anesthetized with 1% isoflurane. Mouse heart rates were in the near physiological range, preventing confounding of diastolic dysfunction measurements by heart rate changes. Transthoracic echocardiography was performed using a Sonos 5500 ultrasound unit equipped with a 15-MHz linear-array transducer and a 12-MHz phase-array transducer. Now, we have available a VisualSonics Vevo 770, too. LV diastolic function was evaluated by the conventional pulsed-wave Doppler recording of mitral inflow velocities (E and A) and two of the newer, relatively load-independent, echocardiographic indices of diastolic function: LV inflow propagation velocity (V_{P}) by color M-mode Doppler, and mitral annulus longitudinal velocities (E′ and A′) by pulsed-wave tissue Doppler imaging (FIG. 9). In our hands, transient occlusion of inferior vena cava (IVC) had little effect on LV inflow propagation velocity or mitral annulus longitudinal velocities. We have shown a strong correlation of echocardiographic and invasive hemodynamic measures under our experimental circumstance, and the proposed studies using both modalities will help validate echocardiographic measures that may prove more expedient in future work.

Hypertensive Mice Show Diastolic Dysfunction by Invasive Hemodynamics:

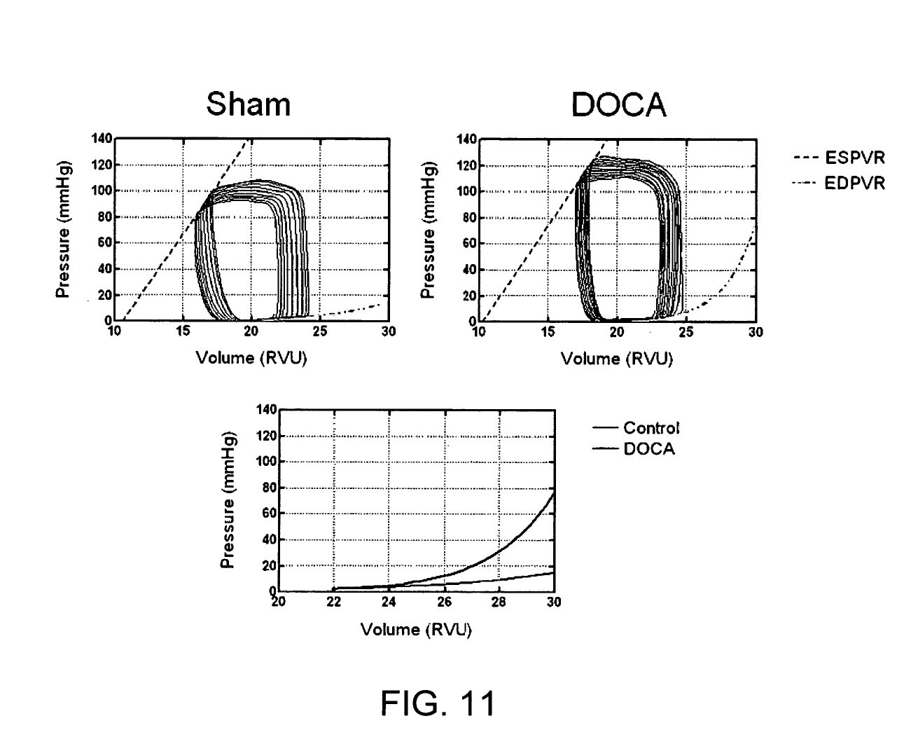

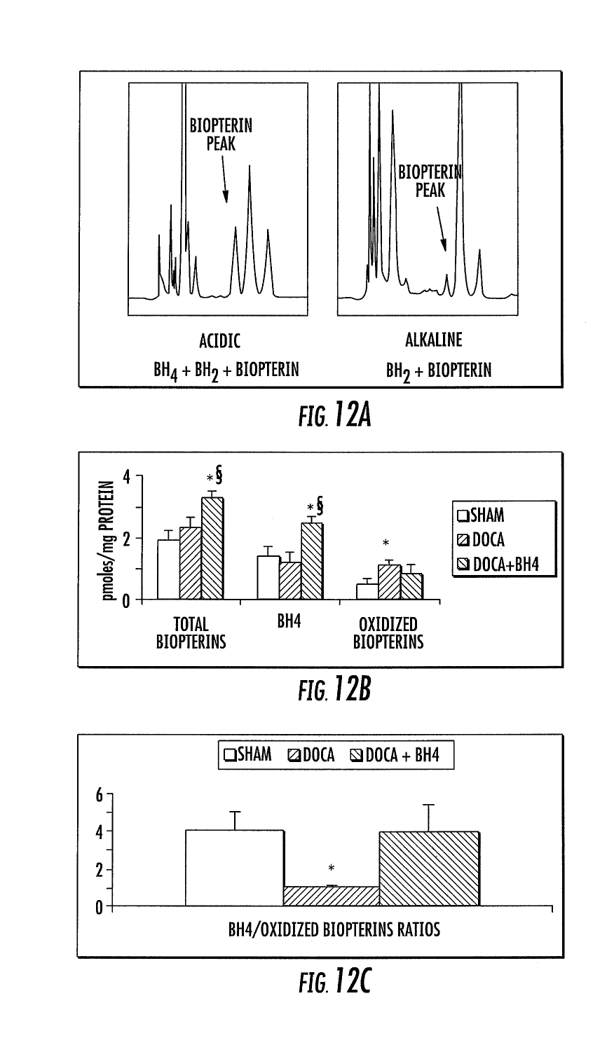

To confirm diastolic dysfunction in our animal model, we have performed invasive hemodynamic measurements, similar to those we published using another mouse model. In the DOCA-salt hypertensive mice and sham-operated control mice, we measured the maximal slope of the LV pressure rise during systole (dP/dtₘₐₓ), the maximal slope of the LV pressure decline during diastole (dP/dtₘᵢₙ), and the LV end-diastolic pressure (LVEDP). Mice were anesthetized with 1% isoflurane and ventilated. Body temperature was maintained at 36.5-37° C. A 1.4-Fr Mikro-Tip catheter pressure transducer (Millar Instruments, Houston, Tex.) was inserted into the right common carotid artery and advanced through the aortic valve into the LV for continuous LV pressure measurements. The catheter was calibrated using an external analog manometer. Data were recorded using a PowerLab system and Chart 5 software (ADInstruments, CO) at a sampling rate of 1 kHz. Each animal was allowed to stabilize for at least 10 min or until stable heart rate, LV systolic pressure, and maximal rate of pressure development (dP/dtₘₐₓ) were observed. Baseline values of heart rate and LV pressures were then recorded for subsequent analysis. As shown in FIG. 10, the DOCA mice had a classic profile for isolated diastolic dysfunction with a significantly elevated LVEDP, reduced dP/dtₘᵢₙ/P (dP/dtₘᵢₙ corrected by corresponding LV pressure), prolonged τ time constant of isovolumic LV pressure decline, and normal dP/dtₘₐₓ/P (dP/dtₘₐₓ corrected by corresponding LV pressure), an index of LV systolic function. Also, we have obtained the end-systolic (ESPVR, an index of LV contractility) and end-diastolic pressure-volume relations (EDPVR, an index of LV stiffness) during inferior vena cava compression, a maneuver to alter preload. FIG. 11 shows that DOCA mice have an increased slope of the EDPVR as compared to sham mice. The findings are consistent with diastolic dysfunction with intact systolic function in DOCA mice.

BH₄ Feeding Prevented the Development of Diastolic Dysfunction in Hypertensive Mice:

BH₄ was pressed into the feed and stored frozen until use as had been done previously by our group. These conditions maintain and effectively deliver systemically BH₄. Dietary supplementation with BH₄, 5 mg/day, beginning the day after nephrectomy, almost entirely prevented the diastolic abnormalities seen in DOCA mice, as shown in Table 1.

Thus, these findings suggest:
1. DOCA-salt hypertension causes diastolic dysfunction in mice, which can be completely reversed by removing the DOCA-salt stimulus.
2. Diastolic dysfunction can be prevented by BH₄ feeding.
   Cardiac BH₄ Content is Decreased in DOCA-Salt Hypertensive Mice:

Tissue biopterin content can be measured using HPLC analysis and a differential oxidation method. We have employed this method in multiple papers to measure BH₄ out of tissues. Recently, we used this method to show that NOX activity is required to oxidize BH₄. With this method, tissue is homogenized in a 0.1 N phosphate buffer at a pH of either 12.0 or 2.0. Pterins at the two pHs are differentially oxidized by exposure to 1% iodine/2% potassium iodide. Using this approach, the non-fluorescent forms of biopterin are oxidized to the aromatic fluorescent biopterin. Under acidic conditions, BH₄ and BH₂ are converted to fully oxidized biopterin. Under alkaline conditions, oxidation of BH₄ results in side chain cleavage and decomposition, while BH₂ is oxidized to biopterin. Thus, the net yield of biopterin from acidic oxidation (BH₄, BH₂ and biopterin) vs. alkaline oxidation (BH₂ and biopterin) can be used to determine the fraction of biopterin in the tetrahydro-form. In FIG. 12, we show that this technique can be used to measure pterin levels in myocardial tissue, oral supplementation increases cardiac BH₄ levels, and that DOCA mice have an oxidative state marked by increased oxidized and decrease reduced pterins. Preliminary studies showed increased cardiac levels of oxidized biopterin (BH₂+biopterin) in the DOCA mice (n=3) relative to the sham-operated mice (n=6). Feeding BH₄ to DOCA mice (n=3) increased both total biopterin content and cardiac BH₄ to above normal level.

The reduction in BH₄ suggested NOS would be less functional, and this dysfunction might contribute to changes in myocardial relaxation. In one test of the idea that the lack of NOS function could contribute to diastolic dysfunction, we studied mice lacking eNOS (eNOS^{-/-}). If the lack of NOS function plays a role in diastolic dysfunction, then it stands to reason that eNOS^{-/-} mice would have diastolic dysfunction. While open to other interpretations, it is consistent with the hypothesis that we found eNOS knockout mice show diastolic dysfunction similar to that in DOCA mice (Table 2).

TABLE 2
Evidence of impaired diastolic relaxation in eNOS^{-/-} mice[0].

| | n | Vₚ | E′ | E′/A′ |
|---|---|---|---|---|
| SHAM | 7 | 45 ± 1 | 5.4 ± 0.3 | 1.7 ± 0.2 |
| DOCA | 7 | 23 ± 1* | 2.9 ± 0.2* | 0.7 ± 0.1* |
| eNOS^{-/-} | 4 | 28 ± 1* | 2.5 ± 0.2* | 0.6 ± 0.1* |

*p < 0.05 vs. SHAM group.

p22^{phox} Knockout Mice to Reduce NADPH Oxidase Activity:

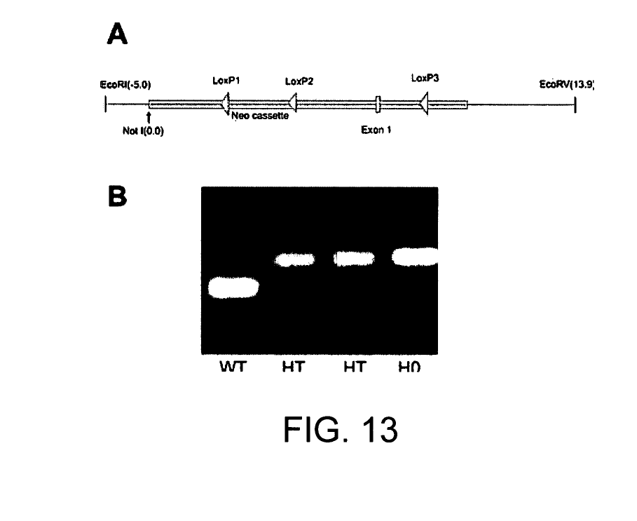

All of the known NOX enzymes, except for Nox5, which does not exist in the mouse, require p22^{phox} as a scaffolding subunit. Therefore, p22^{phox} appears to be an ideal target to prevent activation of all of the NOXs. For this reason, we have created mice in which they have flanked the majority of the coding region of p22^{phox} with loxP sites. To accomplish this, they cloned the mouse p22^{phox} gene and inserted LoxP sites 5′ and 3′ to exon 1. Two 5′ LoxP sites flanking a Neomycin cassette were inserted to allow for negative selection, and a third LoxP site was inserted 3′ to exon 1. The targeting sequences successfully integrated are shown in FIG. 13. These animals are fertile and pass the altered p22^{phox} sequence to their offspring. They have also bred these animals to homozygosity, and the offspring are viable. These p22^{loxP} animals are currently being backcrossed to the C57BL/6 background.

Strain Considerations:

The preliminary data was obtained with C57BL/6 mice.

Summary of Preliminary Results:

This data establishes that hypertension induces myocardial oxidative stress, BH₄ depletion, and diastolic dysfunction. These findings are prevented by BH₄ oral administration. In summary, these results show the motivation for studying oxidative stress and diastolic dysfunction, a plausible hypothesis linking oxidative stress and diastolic dysfunction, and a clinically relevant animal model of diastolic dysfunction that shows evidence of oxidative stress

Therefore, at least the following is claimed:

1. A method of treating or preventing at least one condition, the method comprising:
administering to a host in need of treatment a therapeutically effective amount of tetrahydrobiopterin (BH₄), wherein the condition is selected from: systolic heart failure, diastolic dysfunction, and diastolic heart failure.

2. The method of claim 1, wherein the step of administering BH₄ comprises:
administering BH₄ in a form selected from: a dietary supplement, a composition, a pharmaceutical composition, and combinations thereof.

3. The method of claim 1, further comprising modulating NAD(P)H oxidase activity.

4. The method of claim 3, further comprising modulating NAD(P)H oxidase activity via ACE inhibitors, angiotensin receptor blockers, peroxisome proliferator-activated receptor agonists, and statins.

5. The method of claim 1, wherein the condition is diastolic dysfunction.

6. A method of treating or preventing at least one condition, the method comprising:
administering to a host in need of treatment a therapeutically effective amount of sepiapterin, wherein the condition is selected from: systolic heart failure, diastolic dysfunction, and diastolic heart failure.

7. A method of preserving diastolic function, the method comprising:
administering to a host in need of treatment a therapeutically effective amount of tetrahydrobiopterin (BH₄).

8. A method of preventing generation of reactive oxygen species (ROS), the method comprising:
administering to a host in need of treatment a therapeutically effective amount of tetrahydrobiopterin (BH₄).

9. A method of preventing at least one of the following: generation of reactive oxygen species (ROS) or diastolic dysfunction, the method comprising:
administering to a host in need of treatment a therapeutically effective amount of ebselen and one or more antioxidants selected from superoxide dismutase, vitamins C and E, alpha lipoic acid, tempol and inhibitors of the NADPH oxidase.

10. A method of screening for compounds useful in treating or preventing at least one of: systolic heart failure, diastolic dysfunction, and diastolic heart failure, the method comprising:
constructing an assay to measure generation of reactive oxygen species (ROS);
contacting a host in need of treatment with a compound that prevents generation of ROS;
detecting the effect of said compound on generation of ROS in said assay; and
determining that the compound is a potential target, if said compound reduces or prevents ROS.

11. The method of claim 10, wherein the condition is diastolic dysfunction.

12. A method of screening for compounds useful in treating diastolic dysfunction, the method comprising:
providing a DOCA-salt hypertensive mouse model, wherein the mouse has diastolic dysfunction, wherein the mouse has an intact systolic function, wherein the mouse is characterized by a rapid onset of diastolic dysfunction that is completely reversible, wherein the mouse is characterized by the absence of LV hypertrophy, and wherein the mouse is characterized by the absence of aortic or mitral regurgitation;
detecting the effect of said compound on diastolic dysfunction; and
determining that the compound is a potential target, if said compound reduces or prevents diastolic dysfunction.
